(19) United States
(12) Patent Application Publication
(10) Pub. No.: US 2011/0200578 A1
(43) Pub. Date: Aug. 18, 2011

(54) INTRAVENTRICULAR HEMORRHAGE THROMBOSIS

(75) Inventors: Daniel F. Hanley, Towson, MD (US); Neal J. Naff, Baltimore, MD (US)

(73) Assignee: THE JOHNS HOPKINS UNIVERSITY, Baltimore, MD (US)

(21) Appl. No.: 12/987,859

(22) Filed: Jan. 10, 2011

Related U.S. Application Data
(63) Continuation of application No. 10/509,694, filed on Sep. 26, 2005, now abandoned.
(60) Provisional application No. 60/368,846, filed on Mar. 29, 2002.

(51) Int. Cl. A61K 38/49; A61P 7/02

(52) U.S. Cl. 424/94.64

(57) ABSTRACT

The invention provides methods of treating intraventricular hemorrhage using thrombolytic agents.

### RELATED APPLICATIONS

This application claims the benefit of U.S. Provisional Application Ser. No. 60/368,846, filed Mar. 29, 2002, the entire contents of which are incorporated herein by this reference.

### BACKGROUND OF THE INVENTION

Intraventricular hemorrhages (IVHs) are estimated to complicate the treatment of over 30,000 adult patients in the United States every year who suffer an intracerebral or subarachnoid hemorrhage^{87, 89}. The conventional treatment of this complication is external ventricular drainage, which treats the most dangerous consequence of intraventricular hemorrhage, obstructive hydrocephalus. There is no evidence that external ventricular drainage (EVD) hastens the resolution of the intraventricular blood clot¹⁰⁵. Thus the conventional treatment of IVH is not directed toward attacking the intraventricular hemorrhage itself. This is a potential deficiency in conventional treatment because there is considerable clinical and experimental evidence that intraventricular hemorrhage (IVH) is an independent and significant contributor to morbidity and mortality in patients with intracerebral and subarachnoid hemorrhage^{98, 99, 115}.

IVH occurs in about 40% of primary intracerebral hemorrhage cases and 15% of aneurysmal subarachnoid hemorrhage (SAH) cases^{1,13,17,53}. The incidence of IVH in intracerebral hemorrhage (ICH) is about twice that of SAH; respectively they account for about 10% and 5% of the 500,000 strokes occurring yearly in the United States^{10,13}. Thus an IVH occurs in about 22,000 people every year in the United States. Most recent research supports the assertion that IVH is a significant and independent contributor to morbidity and mortality in both intracerebral hemorrhage and aneurysmal SAH^{1,15,17,43,77,78,82}. Although IVH severely complicates intracerebral hemorrhage; little organized clinical research has been directed at improving the management of IVH.

Brain hemorrhage is the most fatal form of stroke. For ICH with IVH, the reported case fatality rates range from 50 to 58%. 2.78 Treatments of validated efficacy do not currently exist, and data on the removal of blood, the primary pathogenic element, did not exist prior to the instant invention. Accordingly, there is a need in the art for an effective therapy for hematoma removal validated by a phase III clinical trial. Epidemiological evidence strongly supports the significant and independent contribution of IVH to morbidity and mortality after cerebral hemorrhage. The clinical management of this disorder requires a well-defined neurosurgical procedure—external ventricular drainage (EVD)—in addition to 7 to 14 days of integrated neurocritical care including support of respiratory, hemodynamic, and nutritional needs. The current practice standard of external ventricular drainage (EVD), via intraventricular catheter, alone fails to prevent the morbidity and mortality of IVH^{2,19}.

Intraventricular hemorrhage contributes to morbidity in three ways. First, IVH organizes into ventricular blood clots, which then block the narrow ventricular CSF conduits, producing acute obstructive hydrocephalus. If untreated, obstructive hydrocephalus invariably elevates intracranial pressure (ICP) and, as the increased ICP approaches the arterial perfusion pressure, can quickly progress to death. After IVH, obstructive hydrocephalus is the greatest and most immediate threat to life. Present treatment of IVH-associated obstructive hydrocephalus is to use EVD through an intraventricular catheter (IVC). EVD lowers ICP immediately, but it must be continued until the ventricular blood clots have dissolved sufficiently and CSF circulation is normalized. To date, the role of direct mass effect from ventricular distention has not been well defined. Clinically, controlling ICP does not usually improve the patient immediately². Thus, direct mass effect of IVH may be a significant pathophysiologic event independent of the ICP elevation.

Second, prolonged presence of IVH clot deep within the brain is associated with both mortality^{2,60,72} and decreased level of consciousness^{62,75}. EVD does not consistently improve either one. It does not alter either ventricular size, edema, or the inflammation provoked by the presence of intraventricular blood³³. EVD does not change the time required for blood clot resolution⁵⁷. Indeed, EVD may worsen this edema and inflammation because it is frequently complicated by meningitis. Until now, reducing the size of the intraventricular clot and decreasing the time that deep brain structures are exposed to clot have not been directly addressed by any current IVH treatment.

Third, blood degradation products carried to the arachnoid granulations by the CSF flow may contribute to morbidity. With prolonged contact between blood degradation products and the arachnoid, the ensuing inflammatory response may permanently occlude and scar the arachnoid granulations where CSF is absorbed^{8,18,20,39}. These occlusions in the arachnoid granulations gradually produce delayed communicating hydrocephalus, which, in turn, impairs cognition, gait and balance, and urinary continence. Patients with communicating hydrocephalus require permanent implantation of a shunt for CSF diversion.

The only current therapy for IVH is EVD through an intraventricular catheter (IVC). But, EVD treats only one of the acute consequences of IVH—acute obstructive hydrocephalus. EVD fails to prevent much of the morbidity and mortality of IVH for three reasons: (a) it does not increase the rate of clot resolution; (b) it can be complicated by infection or hemorrhage; and (c) it cannot decrease the degree or incidence of communicating hydrocephalus.

### SUMMARY OF THE INVENTION

The present invention is based, at least in part, on the discovery that administration of thrombolytic agents for the treatment of intraventricular hemorrhage (IVH) is safe, decreases mortality, and accelerates clot resolution

Accordingly, the present invention provides methods for the prevention or treatment of an extravascular hematoma or blood clot in a subject, comprising administering to the subject a therapeutically effective amount of a thromoblytic agent, thereby preventing or treating the extravascular hematoma or blood clot. In preferred embodiments, the blood clot is associated with intraventriclar hemorrhage (IVH), intracerebral hemorrhage (ICH), and/or subarachnoid hemorrhage (SAH).

In one embodiment, the thrombolyic agent is urokinase. In another embodiment, the thrombolytic agent is t-PA or rt-PA. In a preferred embodiment, the thrombolytic agent is administered in conjunction with EVD.

In a preferred embodiment, the thrombolytic agent is first administered between about 12-24 hours after diagnosis of intraventricular hemorrhage, intracerebral hemmorhage, and/or subarachnoid hemorrhage. In another embodiment the thrombolytic agent is first administered between about 24-48 hours after diagnosis of intraventricular hemorrhage, intracerebral hemmorhage, and/or subarachnoid hemorrhage.

In another embodiment, the methods of the invention further comprise performing CT scans at intervals of about 6-24 hours to monitor blood clot size and/or monitor whether bleeding is occurring.

In one embodiment, the thrombolytic agent is administered at least about every 4 hours. In another embodiment, the thrombolytic agent is administered at least about every 5, 6, 7, 8, 9, 10, 11, or 12 hours.

In another embodiment, administration of the thrombolytic agent is stopped when the blood clot size is about 80% of its original size. In a preferred embodiment, the blood clot reaches 80% of its original size about 3 days after the first administration of the thrombolytic agent.

In another embodiment, the methods of the invention use urokinase which is administered in doses of about 5000-50,000 units. In anther embodiment, the urokinase is administered in doses of about 12,500 units.

In still another embodiment, the methods of the invention use t-PA or rt-PA which is administered in doses of about 0.1-10 mg. In another embodiment, the t-PA or rt-PA is administered in doses of about 3 mg.

Other features and advantages of the invention will be apparent from the following detailed description and claims.

### BRIEF DESCRIPTION OF THE DRAWINGS

FIG. 1 depicts scatter plots for each treatment group (control and urokinase-treated) that demonstrate the percentage of initial clot remaining over time for all subjects.

FIG. 2 depicts a graph demonstrating the average percentage of initial clot remaining over time for the two treatment groups (control and urokinase) based on a “synthetic cohort” with equal proportions of males and females (50% in each treatment arm). The estimated mean time to achieve a clot 50% of its original size is faster for the UK group (5.60 days) than for the placebo group (8.54 days)

FIG. 3 depicts a graph showing the mean time (in hours) for various diagnoses and treatment steps in subjects in the rt-PA study.

FIG. 4 depicts a graph showing the outcomes of the rt-PA study.

FIG. 5 depicts a graph of the relative IVH volume (determined by diagnostic CT) versus time in subject 104-004.

FIG. 6 depicts a statistical model for the relationship between clot lysis and consciousness level for patients with an initial ICH volume of 13. FIG. 7 shows the model for patients with an initial ICH volume of 0. Both of these models show that the faster the rate of clot resolution, the higher the predicted GCS score

FIG. 7 depicts a statistical model for the relationship between clot lysis and consciousness level for patients with an initial ICH volume of 0.

FIG. 8 depicts a table comparing the data for several urokinase studies and the randomized, still blinded, rt-PA study

### DETAILED DESCRIPTION OF THE INVENTION

The present invention is based, at least in part, on the discovery that low dose administration of thrombolytic agents for the treatment of intraventricular hemorrhage (IVH) is safe, decreases mortality, and accelerates clot resolution.

The data presented herein show a very large overall benefit accrues to the IVH patient when treated aggressively using a thrombolytic agent. Absolute reduction of 30-day mortality is 50-60%, when actual mortality is compared to severity adjusted, predicted mortality in both cohorts.

Blood clotting initiated when wounded or damaged cells display a surface protein called tissue factor (TF). Tissue factor binds to activated Factor 7. The TF-7 heterodimer is a protease with two substrates: Factor 10 and Factor 9. Factor 10 binds to and activates Factor 5. This heterodimer is called prothrombinase because it is a protease that converts prothrombin (also known as Factor II) to thrombin. Thrombin has several different activities, including proteolytic cleavage of fibrinogen (also referred to as “Factor I”) to form soluble molecules of fibrin and a collection of small fibrinopeptides, and activation of Factor 13, which forms covalent bonds between the soluble fibrin molecules, converting them into an insoluble meshwork—the clot.

Blood clots can be broken down, however through the use of “thrombolytic agents”. As used herein, a “thrombolytic agent”, also referred to as a “thrombolytic compound”, is an agent which is capable of inducing a blood clot to dissolve, break up, and/or solubilize. Under normal conditions, plasma contains plasminogen, which binds to the fibrin molecules in a clot. Nearby healthy cells release tissue plasminogen activator (t-PA), which also binds to fibrin and activates plasminogen, forming plasmin. Plasmin (a serine protease) proceeds to digest the fibrin, thus dissolving the clot. Accordingly, in a preferred embodiment, a thrombolytic agent of the present invention is tissue plasminogen activator, also referred to herein as “t-PA” (or “TPA”). Recombinantly expressed t-PA is referred to herein as “rt-PA”. The methods of the invention preferably use it-PA, but those of skill in the art will recognize that the methods are not limited thereto. rt-PA is available commercially from Genentech (South San Francisco, Calif.) under the name Alteplase® and/or Activase®. Alteplase® is a sterile, lyophilized preparation intended for intravascular infusion. Alteplase is available in 2 mg and 50 mg vials. The powder is reconstituted with sterile water to yield a solution that contains about 1 mg of Alteplase® per mL. The rt-PA is prepared in a syringe that will be used to deliver to the patient. Descriptions of rt-PA can be found, for example, in U.S. Pat. Nos. 4,766,075, 4,777,043, 4,853,330, 4,908,205, as well as many others. rt-PA has been approved by the FDA for intravascular thrombolytic treatment of stroke and myocardial infarction.

In another embodiment, a thrombolytic agent used in the methods of the invention is urokinase. Urokinase is an enzyme which is capable of dissolving blood clots. It is available commercially from Microbix (Toronto, Ontario, Canada) under the trade name ThromboClear™, as well as from Abbott Laboratories (Abbott Park, Ill., USA) under the trade name Abbokinase™. Urokinase is also described in U.S. Pat. Nos. 3,930,944, 3,930,945, and 3,957,582, as well as many others.

In another embodiment, a thrombolytic agent used in the methods of the invention is streptokinase, which is available commercially. However, as a non-human protein, streptokinase may induce an immune response in human patients.

The methods of the present invention provide surprising results, because the use of thrombolytic agents is generally discouraged for treatment of conditions associated with bleeding (Adams, H. P. et al. (1996) Circulation 94:1167-1174; Broderick, J. P. et al. (1999) Stroke 30:905-915; The Quality Standards Subcommittee of the American Academy of Neurology (1996) Neurology 47:835-839). Indeed, it is counterintuitive to administer a thrombolytic agent to treat a disorder caused by bleeding. The package insert provided with rt-PA further states that it should not be used if the patient has experienced bleeding.

The methods of the invention are useful for treating extravascular hematomas and blood clots in a human subject. As used herein, the term “extravascular” includes a hematoma or blood clot that is found outside the vasculature, e.g., in the intraventricular space in the brain. The instant methods are directed to the use of a therapeutically effect amount of a thrombolytic agent. As used herein the term “therapeutically effective amount” of a thrombolytic agent includes an amount sufficient to provide a therapeutic benefit to a patient in need thereof. Therapeutic benefit may be determined by any of the methods described herein, and include, but are not limited to, decrease in blood clot size or volume, decrease in ICP, improvement in GCS, improvement in neurological function, and a decrease in predicted mortality. A therapeutically effective amount of a thrombolytic agent includes the parameters of both dosage amount (e.g., amount of thrombolytic agent administered at one time) and dosage interval (e.g., how often the thrombolytic agent is administered. In a most preferred embodiment, a therapeutically effective amount of a thrombolytic agent is an amount sufficient to reduce the blood clot to about 80% of its original size.

An evaluation of rt-PA pharmacokinetics was performed in a group of 10 subarachnoid hemorrhage patients. This study evaluated single bolus doses of 10, 5 and 0.5 mg rt-PA delivered to the basal cisterns via a non-draining catheter system⁸³. A T_{1/2} of two to three hours was defined. Larger bolus doses (10 and 5 mg) were associated with minor local wound bleeding near the catheter. No serious intracranial bleeding occurred at any dose evaluated. Because of rapid CSF elimination kinetics, a dose interval of every eight hours was evaluated in five patients. For this interval, in a non draining system, a dose of 0.5 mg produced continuous elevation of rt-PA above the 6 μmg/mL therapeutic range^{14,83}. Accordingly, in a preferred embodiment, rt-PA is administered in doses of about 3 mg. In other embodiments, rt-PA may be administered in doses of about 0.1, 0.25, 0.5, 0.75, 1.0, 1.5, 2.0, 2.5, 3.0, 3.5, 4.0, 4.5, 5.0, 5.5, 6.0, 6.5, 7.0, 7.5, 8.0, 8.5, 9.0. 9.5, or 10.0 mg. In still other embodiments, urokinase is administered in doses of about 5000, 6000, 7000, 8000, 9000, 10,000, 11,000, 12,000, 12,500, 13,000, 14,000, 15,000, 17,500, 20,000, 22,500, 25,000, 27,500, 30,000, 32,500, 35,000, 37,500, 40,000, 42,500, 45,000, 47,500, or 50,000 units.

The first IVC injection preferably occurs no sooner than about 12 hours and no later than about 24 hours after the initial bleed and only after confirming appropriate IVC placement by head CT and CSF outflow with normal pressure wave forms. Preferably, no drug injections should be made until 1) about three hours have passed to allow for primary hemostasis after IVC placement and 2) a post-IVC placement CT confirms safe placement. A neurosurgeon or neurocritical care physician or their trained designee should perform IVC injections under standard sterile technique. Injections are preferably isovolemic (i.e. withdrawal volume equals drug plus flush volume). Injections are preferably preceded by gentle aspiration of no more than 5 cc of CSF to minimize ICP elevation. Extracted CSF should be sent to the lab for safety evaluations once a day. Injection of the thrombolytic agent should be followed by a 2-mL flush of normal saline.

Analyses of multiple, daily administration of thrombolytic have been performed on ICH clots treated with UK (10,000 IU) or rt-PA (1 to 5 mg) every six or eight hours. These studies demonstrate a daily reduction of clot volume of 20 to 35 percent per day and reductions of clot volume of 60 to 90% within three days of treatment^{54,66}. Despite injection of thrombolytic directly into the tissue bleeding site, recurrent bleeding occurred at a frequency of 0 to 8% in these series. Multiple daily injections did not lead to infection. Thus, multiple daily doses appear safe and produce a more sustained elevation of rt-PA in the brain. This is associated with rapid and near-complete clot volume reduction. Accordingly, the thromobolytic agent used in the methods of the invention may be administered about every 4, 5, 6, 7, 8, 9, 10, 11, 12, 13, 14, 15, 16, 17, 18, 19, or 20 hours.

In a preferred embodiment, patients are monitored during treatment with the thrombolytic agent by interval CT scans. Such monitoring is done, preferably, every 6, 7, 8, 9, 10, 11, 12, 13, 14, 15, 16, 18, 19, 20, 21, 22, 23, or 24 hours. Interval CT scan monitoring will show whether continued bleeding or rebleeding is occurring. If there is bleeding, the administration of the thrombolytic agent (which would increase bleeding) can be stopped, either permanently, or until bleeding has stopped. Interval CT scan monitoring will thereby decrease mortality by preventing adverse complications, including death, resulting from administration of a thrombolytic to a patient with ongoing bleeding.

Preferably, the thrombolytic agent is administered via intraventricular catheter injection, or via any other method known in the art that is able to administer the thrombolytic agent such that it comes in contact with the clot.

Preferably, the thrombolytic agent is only administered if the catheter has access to the intraventricular clot via the ventricular CSF space. Before dosing, the person administering the thrombolytic agent should view the most recent CT scan and determine that the catheter remains in the ventricle, and that the blood clot remains within the ventricle and in direct contact with the compartment in which the catheter is placed. This provides for additional safety, because the thrombolytic agent is delivered in situations in which the CSF spaces are dosed contain clot that can be lysed. Thus, the delivery of drug will not occur after clot is fully lysed. Similarly, this will further protect patients from unnecessary opening of the IVC, which could increase the infection risk.

The half-life of rt-PA in the cerebrospinal fluid (CSF) is two to three hours. rt-PA is immediately bound to the fibrin clot, but studies demonstrate that free rt-PA exists for some time after administration in the ventricular system. Bulk CSF flow is much slower than blood flow, thus the CSF half-life is significantly longer than the 26.5-minute half-life in the terminal elimination phase of rt-PA in the peripheral arterial circulation; but it is shorter than the 12-hour dosing regimen. The optimal method of delivering a drug with a short half-life is by constant infusion, which is used most often for rt-PA in the peripheral and coronary circulation (AHFS Drug Information). Constant infusion of any agent into the CSF, however, poses several difficult problems, including risk of elevation of intracranial pressure (ICP), as well as ventriculitis. Thus, intermittent isovolemic injections are the safest route of administration.

Unless otherwise indicated, the instant invention uses standardized neurosurgical definitions and clinical protocols.

NACABI consortium criteria for EVD drain placement are: 1) altered mental state; 2) obstructive hydrocephalus; and 3) neurosurgical review of neurological exam and CT scan for consistency with the clinical diagnosis of obstructive hydrocephalus. The decision to place the EVD in an optimal location are preferably made for each patient individually by treating physicians.

Intracranial pressure and cerebral perfusion pressure are preferably monitored before, during, and after the injection. After injection, the IVC is preferably closed for about 1 hour to prevent drainage of the thrombolytic agent away from the clot and to allow adequate time for thrombolytic-clot interaction. The IVC can be reopened within that initial hour if necessary to control medically refractory ICP elevation. Medically refractory ICP should be treated by a standardized regimen of hyperventilation, diuresis, and pharmacological sedation before opening the IVC prematurely. After about 1 hour of closure, the IVC is opened with an appropriate drainage gradient. ICP should be measured every four hours, or more frequently, as clinically indicated.

A daily record of Glasgow Coma Score, blood pressure, ICP, and CSF drainage may be used to assess the patient's clinical response to treatment. The results of daily complete blood cell (CBC) counts, CSF cell counts, protein, and glucose may also be recorded, as well as the length of the ICU stay. Mortality at three and six months may be assessed, along with the other secondary endpoint, degree/incidence of hydrocephalus. The Barthel Index, extended Glasgow Outcome Scale and Modified Rankin scales are preferably used to assess clinical outcome at three and six months.

Preferably, adequate spontaneous cerebrospinal fluid (CSF) circulation and resorption must be restored before the IVC can be safely removed. CSF resorptive capacity is be gauged according to CSF drainage rate. Adjusting the height of the drainage system drip chamber controls the rate of external CSF drainage, and hence the ICP that must be exceeded before the drainage occurs. The drip chamber is usually raised in 5-mm Hg steps every 12 to 24 hours. As this is done, the CSF circulatory pathways and resorptive mechanisms are gradually challenged. If CSF circulation and resorption are insufficient, most of the CSF will continue to drain through the IVC, but if CSF resorption is sufficient, little CSF will drain externally. CSF resorption is usually considered inadequate if more than 200 to 250 cc CSF per day drains through the IVC with the drip chamber set at 15 mm Hg. When less than 100 cc of CSF drains per day, CSF drainage should be stopped and ICP should be monitored for 24 hours as final confirmation that spontaneous CSF resorption is adequate and ICP will not rise to dangerous levels. If ICP stays in an acceptable range, and there is no neurological deterioration, the IVC can be removed. If ICP increases in a sustained manner above 30 mm Hg or there is neurological deterioration, the IVC should be reopened for further drainage or shunt surgery can be elected.

Preferably EVD is discontinued using the specific NACABI protocol detailed above.

Preferably, the thrombolytic agent injections continue as defined by the specific dosing tier for, e.g., about three or four days, unless EVD is discontinued or an endpoint of clot lysis is reached (i.e., 80% clot lysis or a side effect endpoint). In another embodiment, EVD is discontinued when the patient tolerates 24 hours of IVC closure with no sustained elevation of ICP above 15 mm Hg. This criterion is specific, and it represents a widely accepted clinical standard.

In a preferred embodiment, patient care standards require the restoration of adequate, spontaneous CSF circulation before removal of the drain. Without restoration of CSF circulation, IVCs should be replaced as needed. Premature replacement of the IVC is defined as replacement of an IVC earlier than six days because of catheter occlusion.

The goal of IVH thrombolytic therapy is the restoration of normal CSF flow; however, in a preferred embodiment, the resolution of intraventricular clot as determined by CT is an endpoint for drug administration. Thus, thrombolytic agent administration will not continue if IVC is required for management of ICP after resolution of blood clot. Similarly, additional ICH or IVH bleeding, a disseminated systemic bleeding event, the occurrence of bacterial ventriculitis, or in the opinion of the investigator any rt-PA-associated event will be considered an endpoint for drug administration.

Two distinct volumes, the ventricles and the clot within the ventricles, can be determined by modification of a method described by Steiner et al. for computing volumes from axial CT scans⁷⁴. Computer software is then used to determine the pixel count within the cross-sectional area of interest (ventricular system and/or intraventricular clot) outlined by a four-button cursor on a backlit digitizing tablet (Numonics, Montgomeryville Pa., model A56BL with Macintosh Accessory Kit). The count is then multiplied by the area per pixel to obtain the actual cross-sectional area of the region of interest within that slice. The volume of the area of interest within each CT slice is the product of this area and the collimation width of that particular CT slice. The total volume of interest is the sum of the individual volumes of interest within all the slices.

The primary outcome measure of the methods of the instant invention is the percent rate of intraventricular clot lysis. The time of each scan (T_{X}) is determined, to the hour, from the time of the baseline head CT scan (time 0=T₀). The baseline head CT scan is defined as the initial head CT scan performed within the 24 hours immediately before the first drug administration. The absolute intraventricular hemorrhage volume (Vt) present at each CT scan at time t is standardized as a percentage of the initial volume (% Vt) by use of the equation % Vt=(Vt/Vo×100), where Vo is the volume of the hemorrhage on the baseline CT scan. Clot radiographic density is made on CT scans acquired daily. Houndsfield units are used to assess clot density at the central region and at the periphery of the clot.

The incidence of hydrocephalus is a secondary outcome. There are no universally accepted criteria for the timing of and indications for shunt placement for the treatment of hydrocephalus; however, the following criteria for shunt placement may be used:
a) Presence of radiographic hydrocephalus (defined below) associated with one or more of the following clinical findings
   i) Obtundation
   ii) Incontinence
   iii) Gait disturbance

Or
b) Failure to wean from the IVC because of elevation of resting ICP above 20 mm Hg when the IVC is clamped for a 24-hour period.

The head CT obtained most proximate in time to the placement of the shunt is independently evaluated to determine if the subject meets the radiological definition for hydrocephalus (see criteria below). Likewise, the clinical case reports are independently reviewed to determine that shunted patients meet the clinical definition of hydrocephalus (criteria a or b, immediately above).

The degree of hydrocephalus may be determined as described by LeMay et al⁴². The total width of the frontal horns (FH) of the lateral ventricles at their widest point are demarcated by a neuroradiologist and measured. The internal diameter (ID), through both caudate nuclei of the skull (from one inner table of the skull cortex to the other inner table) at that level are similarly determined. The degree of hydrocephalus is considered the ratio of the frontal horn width and inner table width (FH/ID). To determine the incidence, communicating hydrocephalus is considered present if that ratio is above 0.50.

In one embodiment, both the degree of hydrocephalus present in each patient and the incidence of hydrocephalus are determined. The degree rather than the incidence of hydrocephalus is the preferred secondary outcome measure, because the degree controls for the high likelihood that the ventricular system is already enlarged at the time of the initial head CT due to the acute effects of the hemorrhage. The incidence as measure could result in an underestimate of the calculated incidence of hydrocephalus in both groups, thus decreasing the power of the study to detect a significant difference in the incidence. The degree of hydrocephalus, on the other hand, offers a continuous variable for analysis and thus will increase the power of the study to detect a difference. Furthermore, the degree of hydrocephalus is altered by treatment in animal models.

Any patient having a ventriculoperitoneal, ventriculopleural, ventriculo-atrial or lumbar-peritoneal shunt is considered as meeting the operational definition of shunt catheter and therefore to have hydrocephalus. The presence of communicating hydrocephalus is operationally defined as the presence of a shunt catheter on the follow-up head CT scan obtained between days 28 and 32, and at six months. For purpose of analysis, patients with shunts as well as those meeting the criteria for shunt placement are combined and considered to represent the incidence of hydrocephalus. Patients with shunts present between days 28 and 32 and at six months, or who meet the predefined criteria for shunt placement, are considered to have hydrocephalus for the purposes of analysis. This analysis may be repeated at six months. Incidence of hydrocephalus in the two treatment arms may be compared using logistic regression.

Problems with Present Clinical Management of IVH

EVD alone is often inadequate therapy for obstructive hydrocephalus. Although intended to treat obstructive hydrocephalus, EVD is often inadequate in the setting of IVH because the catheter becomes occluded with blood clots. Conventional therapy for catheter occlusion with blood is removal of the occluded catheter and insertion of a second catheter in another location, preferably one that is free of blood. Relocation of the IVC carries about a 1% risk of intracranial hemorrhage^{7,67}. and is often unsuccessful because only a portion of the ventricles can be reliably accessed by an IVC. If the accessible portion is occupied by blood, the new IVC will likely occlude. Thus for many patients, EVD is unsuccessful and they succumb to inadequately treated obstructive hydrocephalus. Even when obstructive hydrocephalus is amenable to EVD, persistent blood clots increase the time that drainage is needed, thus increasing the risk of ventriculitis. The risk of ventriculitis from infection is about 10% to 20% and appears to be directly related to duration of IVC placement^{34,49,69}.

External ventricular drainage does not speed clot resolution. External ventricular drainage of CSF is indicated in patients with IVH to relieve any associated hydrocephalus. EVD cannot, however, remove the clot or relieve local tissue compression from distended ventricles. EVD does not alter the rate of blood clot resolution⁵⁸. Although helpful in controlling increased ICP until the occluding clots are cleared in the CSF conduits by the patient's own clearance mechanism, EVD does not shorten the time that the blood clot is in contact with the ventricular system and deep brain structures. Thus, EVD alone does not alter the impact of intraventricular clot on deep brain tissue.

EVD fails to decrease the degree and incidence of communicating hydrocephalus. Since EVD does not hasten the resolution of the intraventricular blood clot, it does little to prevent the later pathophysiologic consequence of IVH—communicating hydrocephalus. Delayed communicating hydrocephalus is caused by an inflammatory reaction generated by the break down of blood products^{8,18,20,39}, the intensity of which appears to be related to the amount of blood present and the time that the CSF is exposed to the clotted blood^{3,24,29,38,63,81}. Development of communicating hydrocephalus often is associated with cognitive impairment, urinary incontinence, and gait and balance problems. This requires surgery for shunt insertion, leaving the patient with the lifelong risk of shunt occlusions and infections.

An adjuvant therapy that could accelerate the resolution of the blood clot and thus reduce the clot-related pathological events would be a major advance and potential lifesaver in the clinical management of IVH. The instant invention demonstrates that intraventricular thrombolysis using urokinase or rt-PA successfully accelerates the resolution of clots and decreases morbidity due to IVH.

Clot Formation and Clot Dissolution; Biochemical Analysis of Hemostasis and rt-PA

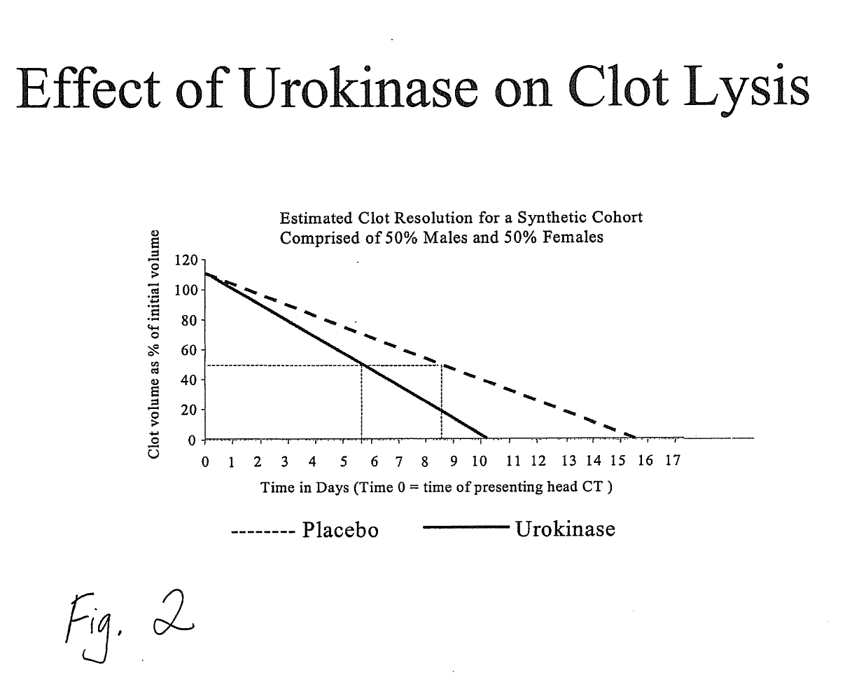

The biochemical events related to clot lysis in the brain's ventricular space are shown schematically in FIG. 2. The normal vascular endothelium maintains blood fluidity. by inhibiting blood coagulation and platelet aggregation and promoting fibrinolysis. The hemostatic system comprises a highly regulated series of procoagulant and anticoagulant zymogens and cofactors. Hemostasis (physiologic response to vascular injury) and thrombosis (pathological formation of thrombus) result from activation of this system. The balance between the coagulation cascade and the fibrinolytic pathway determines the rate of formation and dissolution of the thrombus. Blood coagulation and fibrinolysis are initiated and modulated by compounds embedded in the external membrane of cells (tissue factor, thrombomodulin), deposited in extra cellular matrix (heparin sulfate, dermatan sulfate, protease), or secreted by vascular cells in a regulated manner (von Willebrand factor, plasminogen activators, and plasminogen activator inhibitors).³²

Drug Delivery Routine that Produces Rapid Clot Resolution

For stroke and myocardial infarction, clot resolution usually occurs over minutes, or if not, then during the initial hour of presentation. To accomplish such rapid lysis, constant infusion of drug is carried out either via direct catheter delivery or sustained intravenous delivery. Until now, the data presented herein for intraventricular clot lysis have concentrated on safety, so the amount and frequency of rt-PA administered have not been increased. However, in some embodiments, there are advantages to increasing the frequency of administration. For example, as fresh clot surface is exposed by plasminogen mediated lysis of the clot, new rt-PA can diffuse to previously unexposed plasminogen within the clot, activate it to plasmin, and initiate clot lytic activity at that new site. The ideal time frame for removing clot from the ventricle is probably longer than for stroke or myocardial infarction, but it is most likely a great deal shorter than the 10 to 14 days demonstrated in patients treated with urokinase. For example, in preferred embodiments, clot resolution may occur at about 9, 8, 7, 6, 5, 4, 3, or 2 days.

Dose-Effect Relationship between IVH Volume and Mortality

Prior studies have suggested an independent relationship between the presence of IVH and increased mortality^{11,77-79}. The effect of IVH size on mortality exists independent of ICH hematoma size⁷⁹. A stronger confirmation of the importance of this factor comes from the presence of a continuous relationship between IVH volume and the effect it produces, in this case “mortality”⁷⁸. This direct relationship exists for IVH sizes from zero to about 50 or 60 cc⁷⁹. The increased volume of an IVH within this range accounts for a 50 to 100% increase in mortality. These data strongly support the idea that IVH produces morbidity but is potentially reversible. Accordingly, a method of decreasing the volume of IVH could decrease mortality. The instant invention achieves this goal.

### Natural History of Intraventricular Hemorrhage Resolution

Many investigators have studied the time course of blood clot lysis in CSF by measuring the concentration of various cellular degradation products^{6,30,31,64} and changes in CT scan attenuation^{9,50,59,71}, but no validated, volumetric studies of clot lysis in human brain have been reported. To address this issue we performed a volumetric analysis of intraventricular blood clot lysis in patients with IVH⁵⁸. We validated the accuracy of this method by repeating blinded assessments of clot volume. Intra-observation variability was <1.5%.

The volume of hemorrhage within the ventricular system was determined by digitized volumetric analysis of the head CT scans. For analysis, the time of the initial presenting head CT scan was defined as time 0, and time from the initial head CT to each subsequent head CT scan was calculated. Overall the clot appears to resolve at a uniform rate in terms of percent of initial clot volume (percent clot resolution rate), suggesting that blood clot resolution in CSF follows first-order kinetics (constant percent of substrate conversion/time). Higher order relationships did not provide better data fits.

We further tested this finding by analyzing the resolution kinetics of the individual clots, calculating the average rate of clot resolution (cc/day) for each individual clot. The average rate of resolution varied directly with the initial clot volume, R²=0.88, p<0.001. If clots follow first order kinetics, then percent resolution rates should be constant regardless of the initial clot volumes. To test this theory, we divided the clots at their median volume of 25 cc into large clots and small clots. Mean volume of the large clots was 48 cc and of small clots 14 cc. No significant difference in the percent resolution rate was demonstrated between the two groups.

In statistical analysis of the percent clot resolution rate using cross sectional time-series linear regression, with robust estimates of the standard errors to adjust for clustering of observations, we found no effect on the percent rate of clot resolution from age, gender, presence of intraventricular catheter (IVC), or the type of underlying hemorrhage (intracerebral or subarachnoid). The percent clot resolution rate was estimated as 10.8% of initial volume per day (95% confidence interval, 9.05-12.61%), with a y intercept of 108.3%, as mentioned above. By use of these estimates, the clot half-life (the time at which the volume was estimated to be 50% of the initial volume) would be 5.4 days (95% confidence interval, 4.2-6.7 days). Two factors were identified that did not achieve statistical significance, gender and IVC drainage. Women's clots resolved faster and unexpectedly, IVC use was associated with a slowing of the clot resolution.

From these findings, it can be concluded that: the clot resolves at a constant percent rate consistent with first order kinetics. These findings may suggest that the thrombolytic activity is most likely intrinsic to the clot.

The constant percent WE clot resolution rate for the initial seven to 10 days, in our study, suggests that the interaction of at least two molecules is important to the kinetic understanding of clot lysis. Thus this analysis supports the presence of an enzyme-substrate system (t-PA-plasminogen) critical to clot lysis. That sufficient thrombolytic substrate(s) is present in most clots is also supported by biochemical studies showing that quantities of plasminogen present within a formed clot are sufficient to produce enough plasmin to lyse the clot when activated by plasminogen activator^{5,65}.

### Prospectively Defined Clot Lysis Rate

The data in the urokinase study presented herein (see Example 1) demonstrates: 1) A slow resolution of IVH clot in the untreated patients similar to that found in our natural history study (5.7% per day rate of clot resolution) and 2) An acceleration of IVH clot lysis with urokinase compared to placebo (10.3% per day). This effect is present in the initial 11 subjects. There is a difference of 70 hours seen in time to reach 50% of initial clot volume in the treated patients. This reduction in clot resolution represented the result from a model in which a significant difference was related to both drug and gender, and it is accompanied by shortened drainage time and fewer deaths. Additional findings include the direction and magnitude of clot lysis is similar in both men and women. However, the normalized rate of clot lysis proceeds at about 4.5% per day faster in women than in men. Although the population size does not allow for formal analysis of gender-related effect, this finding is consistent with a previous retrospective analysis⁵⁸ and is similar to the findings of enhanced fibrinolysis in pre-versus postmenopausal women.

### Treatment and Mortality

Further analysis of a large cohort of IVH patients has disclosed a direct and independent relationship between volume of IVH and mortality, which was independent of the size of parenchymal hematoma⁷⁹. An analysis of 30-day mortality comparing actual mortality with predicted mortality for the data presented herein for urokinase (Example 1) and t-PA (Example 2) is presented below in Table I.

TABLE 1
Predicted Probability of Death vs. Actual Death in Patients Receiving
Urokinase/Placebo

| | | | | | | PREDICTED | | P-VALUE |
|---|---|---|---|---|---|---|---|---|
| | | AVG. | | | AVERAGE | PROBABILITY | ACTUAL | FOR |
| | SAMPLE | IVH | AVERAGE | AVERAGE | PULSE | OF | PROBABILITY | MORTALITY |
| STUDY/PATIENT GROUP | SIZE | SIZE | ICH SIZE | GCS | PRESSURE | MORTALITY | MORTALITY | REDUCTION |
| PILOT UK/ALL PATIENTS | 12 | 34.8 | 3.93 | 9 | 92 | 58.00% | 25% | 0.022 |
| RANDOMIZED UK/UK | 6 | 66.8 | 3.71 | 8 | 97 | 38.00% | 0% | 0.057 |
| PATIENTS | | | | | | | | |
| RANDOMIZED | 5 | 41.5 | 13.3 | 8 | 95 | 58.00% | 20% | 0.103 |
| UK/PLACEBO PATIENTS | | | | | | | | |
| (Example 1) | | | | | | | | |
| RANDOMIZED t-PA/ALL | 39 | 52.25 | 8.22 | 8 | 86 | 76.44% | 21% | <0.00001 |
| PATIENTS (Example 2) | | | | | | | | |

This analysis shows a full range of initial presentation severity with a wide range of predicted probability of death, but a very low actual mortality rate at 30 days after presentation. Using previously established disease severity parameters⁷⁷⁻⁷⁹, we compared the mortality predicted by Tuhrim⁷⁹ to the actual mortality observed in both sets of patients. The 25% mortality rate in the open-label group compares quite favorably to the anticipated 58% mortality. Indeed, the probability of observing three or fewer deaths in 12 patients is 0.02 if the underlying mortality rate is 58%. When all our randomized patients are considered, only a single death occurred despite a Tuhrim algorithm-predicted mortality of 47%. The probability of a single death occurring among 11 patients, when the expected mortality is 47% is 0.001. When the randomized patients are divided into the drug treatment groups the difference between predicted mortality and actual was 38% versus 0% for UK treatment and 58% predicted vs. 20% actual for the placebo group. Our current rt-PA group is added for comparison. This group shows a similar finding of approximately 55% difference between predicted and actual mortality. Thus, a robust treatment effect is quite possible. Similarly, the low mortality suggests that the treatment is not overly hazardous.

### Relationship Between Time to Achieve Clot Lysis and Level of Consciousness

In the same urokinase study we collected daily Glasgow coma scale (GCS) scores as a measure of level of consciousness. This analysis demonstrates a slow increase in the score from about eight or nine to about 11 over the first week of urokinase or placebo treatment. The trend and magnitude of improvement is similar in both groups. The size of IVH was similar in both groups, although the average was slightly larger in the treated group. Similarly the size of the associated ICH was small in both the placebo (10 cc) and the treated groups (5 cc).

Analysis of rt-PA Pharmacokinetics from Intraventricular Hemorrhage Treatment

To date, samples from seven patients (6 active treatment and 1 placebo) have been evaluated in the PK portion of the rt-PA study for intraventricular hemorrhage. Results of these analyses are as follows. No rt-PA was identified in the CSF of the placebo patient. Pretreatment levels of rt-PA are below 5 ng/mL in all treated patients. The response after treatment with Dose 1 was evaluable for one patient. This patient's rt-PA displays a first-order pharmacokinetic process with a terminal half-life of 3.1 hours. The concentrations of rt-PA achieved following administration of the 3-mg dose was significantly higher than the concentrations observed of the two major rt-PA inhibitors; PM-1 and α2-antiplasmin, which were essentially undetectable. The plasminogen in this patient showed a time-dependent decline and was undetectable 20 minutes after the dose was administered. The fibrin-split products showed a time-dependent increase with the largest amount (80-160 μg/mL) measured 20 minutes after opening of the intraventricular catheter.

Two patients had data collected after Dose 6 (FIG. 12). These patients showed an initial drop in rt-PA antigen one hour after the opening of the intraventricular catheter (2 hrs. post dose) to levels below that reported to promote fibrinolysis (6.0 μmg/mL) in myocardial infarction¹⁴. The biological activation of fibrinolysis was evident after dosing. These patients had undetectable levels of PAI-1, α2-antiplasmin, and plasminogen prior to Dose 6 and at all time points following administration of Dose 6. Fibrin-split products showed a time-dependent increase after Dose 6 and the increase of fibrin-split product levels were consistent with those measured following Dose 1.

In summary, a 3-mg dose administered into the ventricle achieves prothrombolytic levels of rt-PA (>6.0 μmg/mL) for one to three hours in our patients. ¹⁴This increased rt-PA concentration is associated with increased concentration of fibrin-split products. No inhibitors of fibrinolysis were identified. All endogenous plasminogen was rapidly activated by rt-PA injection. Thus, T_{1/2} data suggests that 12-hour administration produces therapeutic levels about four hours per day or 17% of the time. Accordingly, in a preferred embodiment, the thrombolytic agent is administered about every 4-8 hours to achieve a prolonged and therapeutic elevation of peri-clot rt-PA above about 6.0 μmg/mL.

### Acceleration of Clot Resolution

Until now no data has existed to demonstrate the relevance to humans of the variable “rapid rate of clot removal”. An analysis of the first 28 successfully treated patients now demonstrates two critical findings: 1) Similarity between the animal and human response of consciousness to rapid removal of blood clot. 2) A probable acceleration of IVH clot lysis rate. We grouped the living IVH patients, but excluded the patients with symptomatic rebleeding, because clot reduction was not uniform in these patients. This group includes approximately 50% control and 50% rt-PA treated patients. No inferences can be made in regards to the treatment distribution of deaths and rebleeds from this data since the distribution for all enrolled patients may deviate quite markedly from 50/50. As a group, the mean clot resolution rate was 17.2%, the 75^{th} percentile was 23.7%; the fastest rate was 48.6% per day. Each of these rates is faster than any rate observed in previous studies. They also fall outside of the 95% confidence intervals for the clot resolution rate in untreated IVH patients. They suggest the presence of accelerated clot lysis, possibly related to test article. Thus, the current protocol appears to have achieved some acceleration of clot lysis compared to our previous experience.

Clot Resolution Rate Correlates with Recovery of Consciousness

An analysis of random effects, cross-sectional time series regression analysis of factors independently associated with improvement in level of consciousness during the initial four days of treatment demonstrates that three factors are independently associated with GCS improvement over the initial 96 hours of EVD drainage and “test article” treatment: initial IVH volume, presentation GCS, day of treatment and rate of clot resolution. Only the last is potentially modifiable with treatment. Its modification is the goal of the instant invention. There is a five-point improvement in GCS can be achieved if clot resolution rate is 46% per day vs. no change of GCS with a resolution rate of −2% per day. Thus, the treatment goal of accelerated clot size reduction over 96 hours is associated with beneficial intermediate clinical improvement.

### Safety Review Urokinase: Drug Administration, ICP, and Ventriculitis.

Twelve patients were randomized. One misrandomization occurred (after randomization, but ineligible before the first dose). Six patients from three hospitals were randomized into the UK treatment arm within a 2-year period. The average course of treatment was 3.5 days (range 2 to 6 days). There were no hemorrhagic complications. Treatment was discontinued in two patients, owing to a rising CSF white blood cell count suggestive of ventriculitis. One patient met the criteria for diagnosis of bacterial ventriculitis. These episodes were treated with appropriate antibiotics without clinical sequelae. One patient who entered the study with a 15-cc associated intracerebral hemorrhage died of a cardiac arrest that was deemed unrelated to drug/placebo administration. The remaining patients were all discharged from the hospital to rehabilitation. Two of the patients developed communicating hydrocephalus and were treated with permanent shunts. ICP appeared to be well controlled throughout the protocol. Closure of the IVC drain was tolerated without incident.

Additionally, the analysis of six urokinase treated and five control patients (Example 1) demonstrates an excellent safety profile for the thrombolytic urokinase, including an overall mortality of 8.3%, an 8.3% rate of ventriculitis, and no rebleeds. An absolute benefit of a 20% improvement in mortality for drug-treated patients vs. placebo treated was found. An estimated rate of resolution for the gender-equalized urokinase-treated patients was 11.4% per day compared to 7.8% per day for a similarly gender-equalized control group. This difference in clot resolution rates represents a difference of three days in the time to reach the 50% clot reduction point and a difference of more than four days (from more than 12 to 8 days) to reach the 80% clot size reduction point. In summary, when comparisons to the natural history of ICH are considered, all available data on low dose thrombolytics show that urokinase can be given safely by using intraventricular injection in patients who have recently (within 24-48 hours) experienced severe intraventricular hemorrhage.

Safety of rt-PA

After 39 patients, compliance with dosing and administration was high. Twenty-three of 489 planned doses were not administered for a compliance level of 95%. Evidence of elevated intracranial pressure with system closure did not occur, nor was compromise of cerebral perfusion pressure (CPP) noted. Mean ICP before vehicle administration was 12 mmHg as compared 16 mmHg during the hour after administration. Mean CPP was 85 mmHg before and 85 mm Hg after the same time period. After 45 enrollments, eight deaths have occurred for a mortality rate of 18%. Serious adverse events have been reported in 23 instances. Three episodes of ventriculitis have occurred. None are deemed to have produced permanent disability. An analysis of the relation between number of injections of test article administered and infection does not demonstrate an association between increased dosing frequency and infection. The median number of test article injections is 12; the average is 12.44. This is the same as the first dosing tier and close to the maximal number of injections planned in the other two arms. The maximum number of injections in this study will be equal to the 71st percentile of the current group. None of the patients in the current group receiving 18 or more injections experienced ventriculitis.

Eleven episodes of additional intracranial bleeding have occurred: five of these were clinically symptomatic, two required surgery, and both survived. No unusual adverse events have been noted to date. Event rates for the major safety endpoints, death (17.8%), rebleeding (24.4%) and bacterial ventriculitis (6.7%), remain well below the pre-specified safety thresholds of 75%, 35%, and 30% respectively that represent the natural history of this disease entity.

This invention is further illustrated by the following examples which should not be construed as limiting. The contents of all references, patents, and published patent applications cited throughout this application, as well as the figures, are incorporated herein in their entirety by reference.

### EXAMPLES

### Example 1

### IVH Thrombolysis Using Urokinase

### Patients and Methods

### Patient Selection

Three study centers participated in the enrollment of this study population including, The Johns Hopkins University, Baltimore, Md., The Columbia-Presbyterian Medical Center, New York, N.Y., and University Hospital, Innsbruck, Austria. In addition to Food and Drug Administration Investigational New Drug (BB-IND 5459) approval, the Institutional Review Boards at each of these participating study centers approved the study prior to subject enrollment. The mortality data for the 12 patients presented herein have been previously reported as part of a larger group prior to unblinding of the study drug assignment¹⁰⁴.

Study subjects were patients with a spontaneous intracerebral hemorrhage (ICH) and an associated intraventricular hemorrhage large enough to require external ventricular drainage (EVD) for the treatment of obstructive hydrocephalus. The decision to treat with EVD was distinct from the study protocol and was made by a treating physician prior to enrollment into the study. Therefore, no patient was exposed to the risk of EVD who otherwise would not have received EVD for conventional treatment. Patients or their family members were approached for informed consent only after EVD had been instituted.

Patients were enrolled within 24 hours of the initial hemorrhage. Exclusion criteria included clotting disorders, pregnancy, age less than 18 years, and untreated cerebral arteriovenous malformations or aneurysms. In patients with an atypical presentation for spontaneous ICH, the presence of an aneurysm or arteriovenous malformation was excluded by appropriate diagnostic studies. In order to obtain a more uniform patient population for preliminary analysis of clinical outcome, patients with infratentorial hemorrhages, subarachnoid hemorrhage, and patients with supratentorial hemorrhages with an intraparenchymal volume larger than 30 cc were excluded.

### Blinding and Randomization

Randomization was balanced within centers at the time of patient entry. Patients were randomized between two treatment groups: (1) 1 cc placebo injections of normal saline every 12 hours, or (2) injections of urokinase 25,000 IU/1 cc normal saline every 12 hours. The randomization code was not revealed to the investigators. Randomization sheets were generated by the Investigational Drug Service of the Johns Hopkins Medical Institutions (JHMI) using “BSR Version 4.0” software.

### Drug Administration

Urokinase was commercially available as a sterile, lyophilized preparation intended for intravascular infusion¹⁰⁹. 25,000 IU of urokinase powder was reconstituted with 1 cc of sterile water. The resulting solution was clear and colorless, thus facilitating blinded randomization. The study drug or placebo was prepared in a syringe and delivered to the intensive care unit.

The first IVC injection occurred no sooner than 12 hours and no later than 24 hours after the initial bleed and only after confirming appropriate IVC placement by head computed tomography (CT) scan and/or by cerebrospinal fluid (CSF) outflow with normal pressure wave forms. A neurosurgeon or neuro-critical care physician performed the IVC injections under standard sterile technique. Injections were preceded by gentle aspiration of no more than 5 cc of CSF to minimize intracranial pressure (ICP) elevation. Injection of the study agent was followed by a 3-ml flush of normal saline.

Intracranial pressure and cerebral perfusion pressure were monitored before, during, and after the injection. After injection, the IVC was closed for 1 hour to prevent drainage of the study drug away from the clot and to allow adequate time for drug-clot interaction. ICP elevations occurring while the IVC was clamped were treated with additional sedation or hyperventilation. The IVC was reopened within that initial hour only if necessary to control medically refractory ICP elevation. After the 1 hour of closure, the IVC was reopened with an appropriate drainage gradient.

Study agent injections continued every 12 hours until EVD was discontinued according to pre-specified criteria. EVD was discontinued when the patient tolerated 24 hours of IVC closure with no sustained elevation of ICP above 15 mm Hg. Study drug injections were discontinued if there was prospective identification of radiographic extension of the IVH or ICH, or emergence of a systemic bleeding disorder.

### Evaluation of Clot Resolution

Study subjects had a head CT scan performed before the initial intraventricular injection and then daily for the duration of the study. An additional head CT was obtained between 28 to 32 days from the day of initial ICH. At the completion of the treatment phase, all head CT scans were copied and sent to the JHMI Division of Neuroradiology for blinded volumetric analysis.

A neuroradiologist blinded to the treatment arm demarcated the regions of interest (IVH and ICH volume on all scans). After demarcation of the areas of interest, the intraventricular and intracerebral hemorrhage volumes were determined by modification of a method described by Steiner et al. to compute volumes from axial CT scans¹¹³. Custom software was used to determine the pixel count within the cross-sectional area of interest (ventricular system and/or intraventricular clot) outlined by a 4-button cursor on a backlit-digitizing tablet (Numonics, Montgomeryville Pa., model A56BL with Macintosh Accessory Kit). The count was then multiplied by the area per pixel to obtain the actual cross-sectional area of the region of interest within that slice. The volume of the area of interest within each CT slice is the product of this area and the collimation width of that particular CT slice. The total volume of interest is the sum of the individual volumes of interest within all the slices.

### Data Collection and Analysis

A daily record of Glasgow Coma Score (GCS), blood pressure, ICP, and CSF drainage was made to assess the patient's clinical response to treatment. Mortality at 28 to 32 days was assessed, along with the other secondary endpoint degree/incidence of hydrocephalus. Clinical outcome was assessed between days 28 and 32 by using the Barthel Index and Modified Rankin scales.

The pre-specified primary outcome measure of this study was the rate of radiographic intraventricular clot resolution. The time of each scan (t) was determined from the time of the baseline head CT scan (t=0). The baseline head CT scan is defined as the initial head CT scan performed within the 24 hours immediately before the first drug administration. For these analyses, the time from the baseline scan to the subsequent scans was rounded to the nearest hour.

The intraventricular hemorrhage volume (Vₜ) present at time t was standardized as a percentage of the initial volume (% Vₜ) by use of the equation % Vₜ=(Vₜ/Vₒ×100), where Vₒ is the volume of the hemorrhage on the baseline CT scan, and statistical analysis was carried out on this standardized data.

The duration of EVD is a secondary outcome measure that was evaluated. The duration of EVD was considered as the time between the initial placement of the IVC and the time that the final IVC was discontinued. Daily CSF drainage volume and ICP values were monitored to evaluate compliance with the discontinuation protocol cited above.

### Statistical Methods

Baseline characteristics of the groups with discrete values were compared with Fisher's exact test; continuous base-line characteristics were compared with Student's t-test. The predicted mortality in ICH patients with an IVH was obtained using the logistic regression model developed by Tuhrim et al.¹¹⁵. The 30-day survival probability (P) for a given patient may be calculated from this model using the formula:

$$P = \frac{{e}^{- 3.3125 + 2.7859 GCS + 0.0180 ICH + 0.5832 PP - 0.9567 HYDRO + 0.0979 IVH}}{1 + {e}^{- 3.3125 + 2.7859 GCS + 0.0180 ICH + 0.5832 PP - 0.9567 HYDRO + 0.0979 IVH}}$$

where GCS (Glasgow Coma Scale score) can assume values of 0 (GCS>8) or 1 (GCS 8); ICH=size of intraparenchymal hemorrhage size measured in cm³; PP (pulse pressure) can assume values of 0 (PP 85 mmHg) or 1 (PP>85 mmHg); HYDRO can assume values of 0 (absent) or 1 (present); and IVH represents the size of the intraventricular hemorrhage in cm³. After obtaining the predicted 30-day mortality and the actual mortality rate in the entire cohort, these were compared using the exact hypothesis test for binomial random variables. The probability of obtaining the number of deaths observed, or fewer, under the predicted mortality rate was calculated.

The primary outcome measure, mean percent clot resolution rate per hour, was estimated random effects linear regression. The analyses included all patients who met inclusion criteria and were randomized, and patients were categorized on the basis of intent to treat. We tested for the relationship between drug and percent clot resolution rate per hour excluding initial scans since they exhibit no variability (% Vₒ=100). Prior observations by our group had suggested a possible effect of gender on clot half-life¹⁰³. Thus the pre-specified analysis for drug effect was planned to include gender. Statistical analyses were performed using Stata statistical software (Stata Corporation, College Station, Tex.). All statistical tests were two-tailed with the exception of the comparison of predicted and observed deaths and a P value of less than 0.05 was considered to indicate statistical significance.

### Sample Size

We proposed to test for an absolute effect size of an increase of 3 percent in the percent of blood clot resolved each day. This absolute effect size would represent an approximately 30% relative increase in the percent blood clot resolution rate of 10.8% demonstrated in our retrospective natural history study of intraventricular clot resolution¹⁰⁵. The sample size was calculated by methods suggested by Dawson-Saunders for computation of sample sizes to compare means of a continuous variable for two independent groups⁸⁸. The calculated sample size, assuming an absolute effect size of 3%, a standard deviation of 2.55%, a two-tailed significance level of 0.05, and 90% power, was 15 per group. We proposed to increase the recommended sample size from 30 to 48 to allow for patient heterogeneity and to increase the power of the study to potentially detect a smaller effect size.

### Results

### Patient Demographics

Twelve patients were enrolled into the study at one of the university-based referral centers (Johns Hopkins University, 9 patients; Columbia-Presbyterian Medical Center, 2 patients; University Hospital, 1 patient). Enrollment was terminated when urokinase was withdrawn from the market (although it has since been returned to the market). Eight patients were male, 4 were female, and their mean age was 51.9 (range 36 to 74; SD±11.5) years. Seven patients were enrolled into the urokinase treatment group, and five patients were enrolled into the placebo group. One patient was randomized to the urokinase treatment group but did not receive any drug doses due to the development of an acute upper gastrointestinal hemorrhage after randomization, which was a contraindication to study treatment. That patient is not included in the outcomes analysis because no test agent was administered.

The relevant demographic, clinical and neuroradiologic characteristics of the study cohort are summarized in table 1. The urokinase treatment group and the placebo group were similar in age (49.6 v. 55.2 years, P=0.43), presenting GCS (7.14 v. 8.00, P=0.72), mean baseline ICH volume (5.3 cc v. 13.12 cc, P=0.06), and mean baseline IVH volume (72.8 cc v. 41.54 cc, P=0.07).

### Treatment Characteristics

The urokinase group received a mean of 7.7 doses of urokinase (range 4 to 11) and the placebo group received a mean of 19 doses (range 4 to 36). This difference in dosages was not significant (P=0.12). The placebo group required a longer mean duration of external ventricular drainage (207.8 hours, range 54.3 hours to 351.3 hours) than did the urokinase treatment group (169.8 hours, range 115.0 hours to 278.5 hours) but this difference was not significant (P=0.52). A mean ICP and CPP were calculated for each patient from the ICP and CPP recordings collected every 4 hours for the duration of drug administration. The urokinase and the placebo group had similar mean group ICPs (15 vs. 11 nun Hg) and CPPs (103 vs. 112 mm Hg).

### Intraventricular Clot Resolution Rate

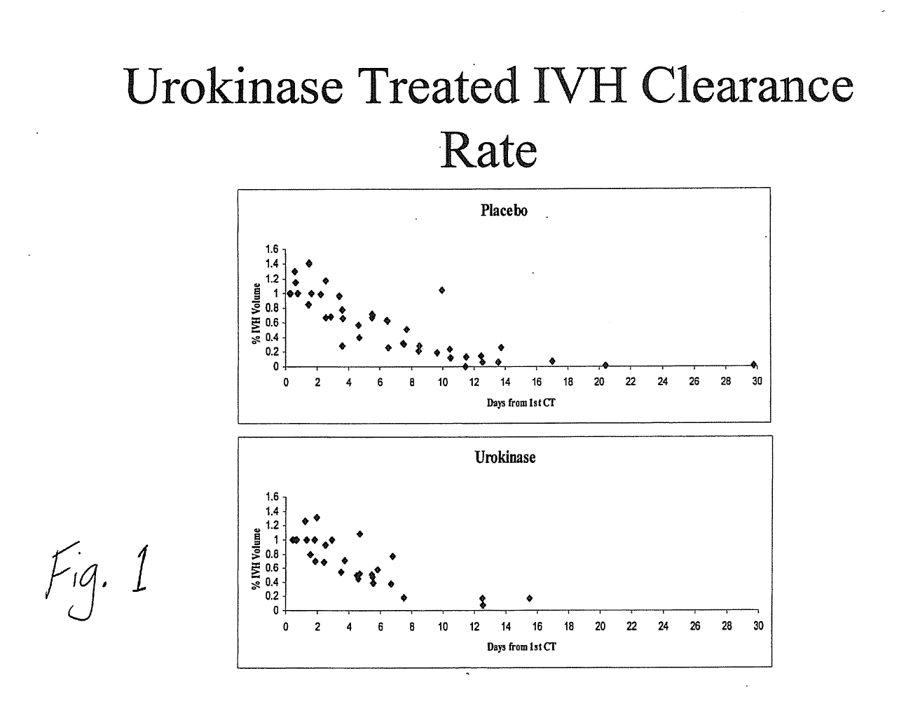

FIG. 1 shows a scatter plot of the percent of initial clot remaining against time since baseline for all subjects by treatment group. Clot resolution rate per hour was estimated using the data for the first 10 days following the baseline CT. This data set consists of 58 scans (an average of 5.3 per patient; range 3 to 9). Two factors favorably affected the clot resolution rate: randomization to the urokinase treatment arm (P=0.008) and female gender (P 0.001). The amount of intraventricular clot, expressed as a percentage of the initial clot volume, that remains at t hours following the baseline head CT scan can be expressed by the following equation:

IVH volume remaining=111.2−(0.231*t)−(0.148*treatment arm*t)−(0.191* gender*t)

where t=time in hours since baseline CT scan

treatment arm=1 if patient received UK, =0 if placebo

gender=1 if patient is female, =0 if male

Clot half-lives (time to a volume 50% of initial volume) based on this equation were 107.4 hours and 161.6 hours for female and male urokinase patients versus 145.0 hours and 264.9 hours for control patients. FIG. 2 demonstrates the average percentage of initial clot remaining over time for the two treatment groups based on a “synthetic cohort” with equal proportions of males and females (50% in each treatment arm). An assumption about the male-female distribution is necessary for such a figure since actual slopes will depend on the male-female proportions. Note that the time to achieve a clot of 50% of its original size for such a cohort cannot be calculated by averaging the gender specific clot resolution rates. Rather, for each treatment group, times to 50% of initial volume were calculated for males and females. These times were then averaged and the rate of clot resolution that would result in that average was calculated. The estimated mean time to achieve a clot 50% of its original size was faster for the UK group (5.60 days) than for the placebo group (8.54 days). This translates into a 2.9 day reduction (34.4%) in the intraventricular clot half-life for this synthetic cohort of UK treated patients. The clot resolution rates that would correspond to these slopes are (10.9%/day, CI 7.7%-14.0%) for the UK treated group and (7.1%/day, CI 4.1%-9.9%) for the placebo group.

### Communicating Hydrocephalus and Adverse Events

Within the entire study cohort only one patient randomized to the placebo arm had developed delayed communicating hydrocephalus at the time of the 30-day follow-up head CT. No meaningful conclusion regarding the effect of intraventricular thrombolysis on the development of delayed communicating hydrocephalus can be drawn from this study.

There was no discernible difference in the number of adverse events in the two groups. Among the entire cohort there was one case of ventriculitis, two cases of bacteremia, and three cases of fever of unknown origin. There were no incidences of expanded intracerebral hemorrhage or intraventricular hemorrhage that were prospectively identified.

### Mortality

There was one death in the placebo group resulting from a cardiac arrest after the subject's family made the decision to change his status to do not resuscitate and to withdraw care. There were no deaths in the treatment group. The predicted mortality for the treatment group using the Tuhrim algorithm described above was 38%. The predicted mortality for the placebo group was 58%. The predicted mortality for the entire study group was 47%. Although the observed mortality was much less than the predicted mortality in both groups, there was no statistically significant difference in the degree of reduction in mortality between the treatment groups (P=0.25). This overall reduction in mortality of the entire study cohort from a predicted mortality of 47% to an observed mortality of 9% was statistically significant (P=0.01).

### Urokinase Effect on Clot Resolution

This prospective double-blinded randomized controlled clinical trial demonstrates that intraventricular injections of urokinase hasten the resolution of intraventricular blood clots in patients with spontaneous intracerebral hemorrhages. The favorable effect of intraventricular injections of urokinase on the pre-specified primary outcome measure of percent clot resolution rate would be a relative increase of 31.1% among a patient population comprised of 50% men and 50% women. Clinically, this favorable effect on clot resolution rate translates into a reduction of 2.9 days in the half-life of intraventricular blood clots among such a patient population. Although the study was weakly powered to demonstrate a statistically significant effect of urokinase treatment on mortality due to truncated patient enrollment, the actual mortality in both study arms was significantly less than the expected mortality. Thus, the favorable effect demonstrated on clot resolution rate appears to accrue without any significant adverse effect on mortality.

### Comparison to Clinical Series

Previous clinical series have reported an apparent beneficial effect of thrombolytic agent (urokinase, streptokinase, t-PA) injections on the resolution of intraventricular blood clots^{85, 86, 90-93, 95-97, 100, 101, 104, 107, 108, 111, 112, 114, 116}. The findings that support support a beneficial effect on clot resolution reported by these studies, however, are limited by the lack of volumetric or quantitative analysis of the blood clot volumes over time. Furthermore, the reported series do not include a control arm in which patients receive injections of a non-thrombolytic agent such as saline. Comparison with a control injection is critical because it is possible that the simple mechanical effect of periodic saline injection into the ventricular catheter and ventricular compartment could hasten the resolution of intraventricular blood clots. The control injections could favorably affect clot resolution by improving the patency of the ventricular catheter or by mechanically disrupting the intraventricular blood clot. This study is the first clinical evaluation of intraventricular clot thrombolysis to volumetrically quantify intraventricular clot resolution and to compare thrombolytic injection to a control injection.

### Comparison to Natural History Study-Effect of Ventricular Drainage

Our group has previously reported a retrospective quantitative analysis of the natural history of intraventricular clot resolution¹⁰⁵. The patients studied in that cohort were followed expectantly without a ventricular drain or were treated with external ventricular drainage without thrombolytic injections. Comparison of that natural history cohort to the present study population is informative. The percent rate of clot resolution in the natural history group was 10.8% (95% confidence interval, 9.05-12.61%). This rate is comparable to the rate estimated for a synthetic population of urokinase treated patients (11.4%/day, CI=8.5%-14.3%) in this study. The rate of clot resolution in the natural history group was nearly 40% faster than the rate estimated for a synthetic population of control patients in this study (10.8% vs. 7.8%). Although there are inherent weaknesses with a comparison to a retrospective natural history cohort, the sizeable difference in clot resolution rates does suggest that external ventricular drainage may in fact retard the natural clot resolution process. Our previous study demonstrated a similar trend toward slower clot resolution in the natural history sub-group that was treated with external ventricular drainage versus the sub-group that did not receive external ventricle drainage¹⁰³. A possible explanation of this adverse effect of external ventricular drainage on clot resolution is suggested by our previous study that supported the hypothesis that the tPA that sustains clot resolution is liberated from the clot itself¹⁰⁵. EVD could thus slow clot resolution by draining away the tPA released from the clot into the CSF. It is thus possible that the treatment effect observed in this study is present because the urokinase injections reverse the slowing of clot resolution caused by external ventricular drainage. A higher thrombolytic dose may be necessary to further speed clot resolution beyond the natural resolution rate that occurs without ventricular drainage.

### Small Effect in Comparison to an Animal Model

Another informative comparison is to the elegant canine study conducted by Pang et al¹⁰⁷. This is the only other study to provide a quantitative comparison of intraventricular clot resolution in subjects treated with thrombolytic injection versus controlled (saline) injections. Pre-clotted blood was injected into the ventricles of 20 dogs. The dogs were then treated with 12-hour interval injections of 20,000 IU of urokinase or 0.2 ml of saline. External ventricular drainage did not occur. Head CT's were obtained daily and the ventricular clot volume was determined. The urokinase treated dogs demonstrated a clot resolution rate that was nearly 15 times greater than the saline treated dogs (0.57 days/ml vs. 7.46 days/ml, P<0.001). In addition the urokinase treated dogs demonstrated a markedly reduced interval to return of normal neurologic status versus the control dogs. The present study demonstrates a much less substantial effect on clot resolution rate and did not demonstrate any appreciable effect on neurologic morbidity or on mortality. This profound difference in effect suggests that the dose of urokinase administered in this study was not optimal. Although biologic differences in intraventricular thrombolysis may exist between canines and humans, other factors are likely more important. The average size of the canine intraventricular clots was 6.9 cc. The average intraventricular clot size in our human study subjects was 54.2 cc. The dosage of urokinase in the canine study was 20,000 IU every 12 hours versus 25,000 IU every 12 hours in the present study. The small dosage relative to the canine dosage used in this study was chosen because the similar dose in the canine model resulted in no hemorrhagic complications, and it was consistent with the dosages used in the clinical series that reported minimal hemorrhagic complications. The lack of significant hemorrhagic complications in this study and the limited effect on clot resolution in comparison to a credible animal model would support a dose escalation in future studies.

The robust neurologic improvement seen in the UK treated dogs versus the control dogs was not seen in this study. An interspecies difference in neurologic resiliency may account for some of disparate recovery rate, but it is probably more important that the UK treated dogs all had a dramatically reduced complete clot resolution time (3 to 6 days) versus that seen in the control dogs (38 to 65 days). The reduction in the clot resolution time seen in the UK treated group in this study was only 31%. Additionally, the entirety of the initial neurologic morbidity (all dogs were in a coma initially) in the canine subjects was caused by the placement of clotted blood in the ventricles through a cannula. The associated surrounding brain injury was minimal. The human subjects had additional morbidity from associated intracerebral hemorrhages. The canine study does suggest, however, that ventricular blood itself may cause considerable neurologic morbidity independent of the damage caused by associated intracerebral hemorrhages. Additional animal models support this suggestion⁹⁹. Furthermore, the canine study suggests that hastening the removal of ventricular blood clots may have a profound impact on neurologic recovery. Future studies of intraventricular thrombolysis should be appropriately powered to detect potential improvement in neurologic outcome associated with hastened intraventricular clot resolution.

### Effect of Gender and Neuro-Intensive Care

An unanticipated finding in this study was the profound impact gender had on clot resolution rate with women demonstrating a clot resolution rate that is nearly twice as fast as men. Indeed, the effect of gender was greater than the effect of urokinase injection. To our knowledge this disparity in intraventricular clot resolution between men and women has not been reported previously and should thus be further evaluated in a larger study population. Additionally, it will be important for future studies of intraventricular thrombolysis to allow for this possible gender difference in calculating an appropriate sample size.

Although both treatment arms had an absolute reduction in actual mortality of 38% versus their respective expected 30-day mortality, there was no statistically significant difference in the degree of reduction in mortality between the treatment groups. The study was powered to detect a substantial difference in the primary outcome measure, the percent clot resolution rate, and was weakly powered to detect any difference in mortality. Nevertheless it is encouraging that the addition of urokinase injections did not result in any apparent increase in mortality. Furthermore, these findings suggest that focused neuro-intensive care management including external ventricular drainage may reduce mortality in patients with intraventricular hemorrhage independent of treatment with intraventricular thrombolysis.

### Future Directions

Although patient enrollment into this study was aborted at 25% of the pre-specified sample size of 48 subjects, the study does demonstrate a statistically significant beneficial effect of thrombolytic treatment on intraventricular clot resolution. This beneficial effect accrued without any increase in morbidity or mortality. In an effort to validate these findings with a larger study, the authors and others are conducting a multi-center study that is evaluating the efficacy of intraventricular thrombolysis with tPA. Similar to the present study, the tPA study is a phase II study and by definition is designed to demonstrate a beneficial effect on a surrogate clinical outcome measure, namely the percent clot resolution rate. If the beneficial effect of intraventricular thrombolysis demonstrated in the present study is validated by the tPA study, additional studies with larger study populations would be necessary to demonstrate that hastened intraventricular clot resolution translates into improved clinical outcomes in important measures such as duration of external ventricular drainage, incidence of hydrocephalus, neurologic morbidity, and mortality. Future studies will also be necessary to determine the appropriate dosing of the thrombolytic agent that provides the greatest efficacy with the least amount of added morbidity. Given the limited interventions available to improve the outcomes of patients with intracerebral hemorrhage, the promising results of this study warrant the proposed scrutiny of additional investigation.

### Example 2

### IVH Thrombolysis Using rt-PA

Study subjects were patients with a spontaneous intracerebral hemorrhage (ICH) and an associated intraventricular hemorrhage large enough to require external ventricular drainage (EVD) for the treatment of obstructive hydrocephalus. The decision to treat with EVD was distinct from the study protocol and was made by a treating physician prior to enrollment into the study. Therefore, no patient was exposed to the risk of EVD who otherwise would not have received EVD for conventional treatment. Patients or their family members were approached for informed consent only after EVD had been instituted.

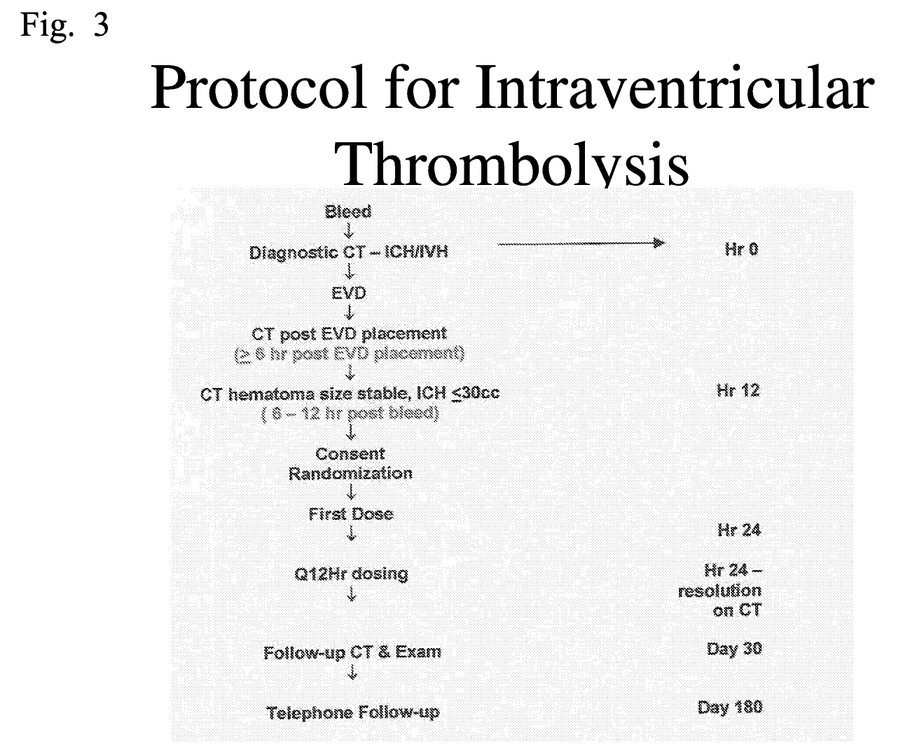

The diagnostic head CT revealing ICH/IVH was designated the starting time point (0 hr post bleed). A head CT was performed after EVD placement (≧6 hr post bleed). Consent was obtained and randomization performed 6-12 hr post bleed, when the hematoma size was stable and ICH≦30 cc by CT. The first dose was given at 12 hours post bleed, and the second dose at 24 hours. Dosing occurred thereafter every 12 hours until clot resolution as seen via CT. A follow-up CT and exam was given on Day 30, and telephone follow-up was performed on Day 180. FIG. 3 shows the mean time (in hours) for various diagnoses and treatment steps.

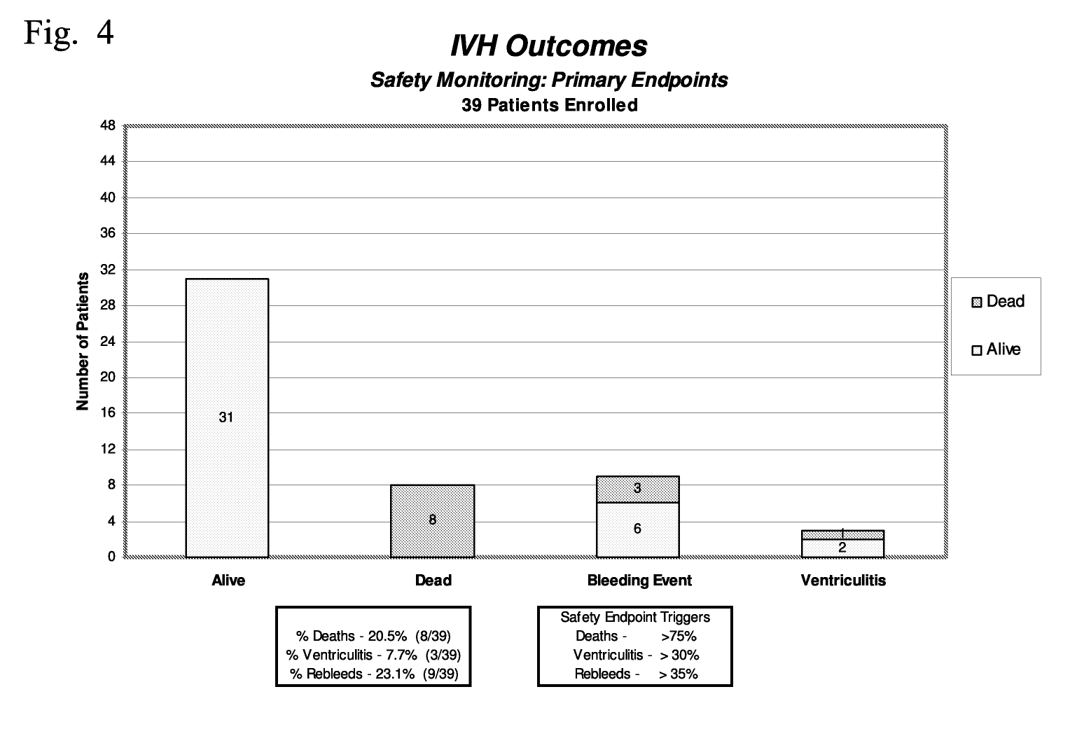

FIG. 4 shows the outcomes of the rt-PA study. Of the 39 subjects enrolled in the study, 20.5% (8 subjects) died, as compared to a predicted death rate when a thrombolytic is not used of about 78%. 7.7% (3 subjects) had ventriculitis, as compared to a predicted rate when a thrombolytic is not used of about 30%. 23.1% (9 subjects) had a rebleeding event, as compared to about 38% when a thrombolytic is not used. It should be noted that these data represent the data for both the rt-PA treated subjects and the control subjects, as the study has not yet been unblended.

Follow-up at 30 days revealed that of the 39 subjects (total of treated and control), 1 had a GOS score of 0 (good), 7 had GOS scores of 1 (moderately disabled), 20 had GOS scores of 2 (severely disabled), 3 had GOS scores of 3 (vegetative state), and 8 had GOS scores of 4 (dead).

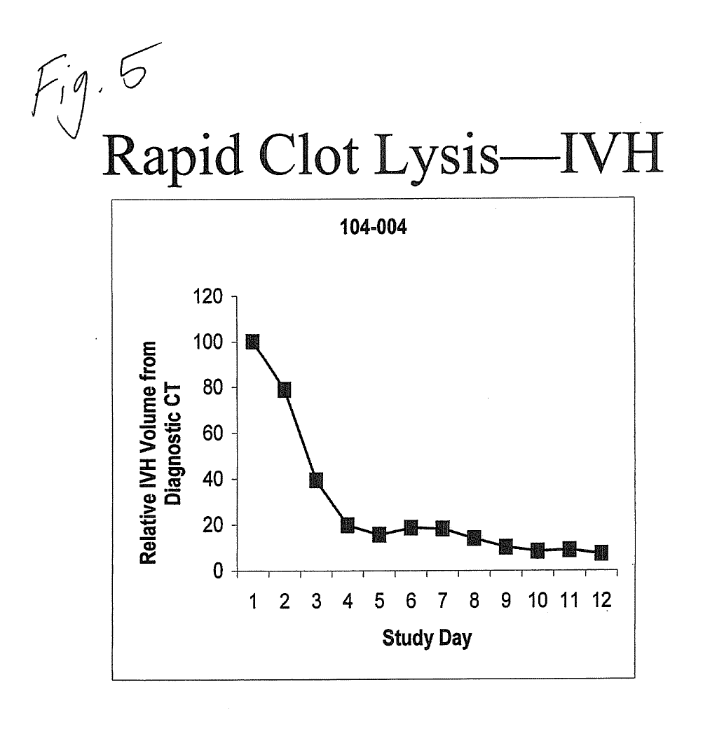

FIG. 5 shows a graph of the relative IVH volume (determined by diagnostic CT) versus time in subject 104-004. This subject shows rapid clot lysis, with 80% clot resolution by days 3-4 of the study.

Overall, this study showed faster rates of clot resolution than those previously recorded for humans. For the first 38 patients, the blood volume removed per day averaged 15.9% of the initial volume. In the patients successfully treated (i.e., no major rebleeds), the blood volume removed per day averaged 17.2% of the initial volume. The fastest rate observed (patient 104-004) was 48.6% blood volume removed per day. The 75th percentile was 23.7% blood volume removed per day. All of these rates are within the 95% confidence level. It should be noted that, because the study has not been unblended, it is not known which subjects were treated with rt-PA and which were controls.

A change in the level of consciousness (measured using the Glasgow Coma Scale (GCS)) was recorded at baseline and at least every eight hours during treatment. A change in the level of consciousness during the first 96 hours was based on a change in GCS score from the baseline level.

In order to do statistical analysis, factors independently associated with a change in the level of consciousness were examined. A random-effects cross-sectional time-series regression was performed for the 28 successfully treated patients. Higher order terms for time were included where significant.

The maximum GCS improvement observed during the 96 hours following baseline GCS had a mean of 3.8 points (standard deviation of 2.9 points). Four independent factors associated with a change in consciousness during the 96 hours following baseline were: time (p<0.001), rate of clot resolution (p=0.02), initial ICH volume (p=0.008), and baseline GCS p=0.001). The model fit the Wald test statistic p-value<0.0001.

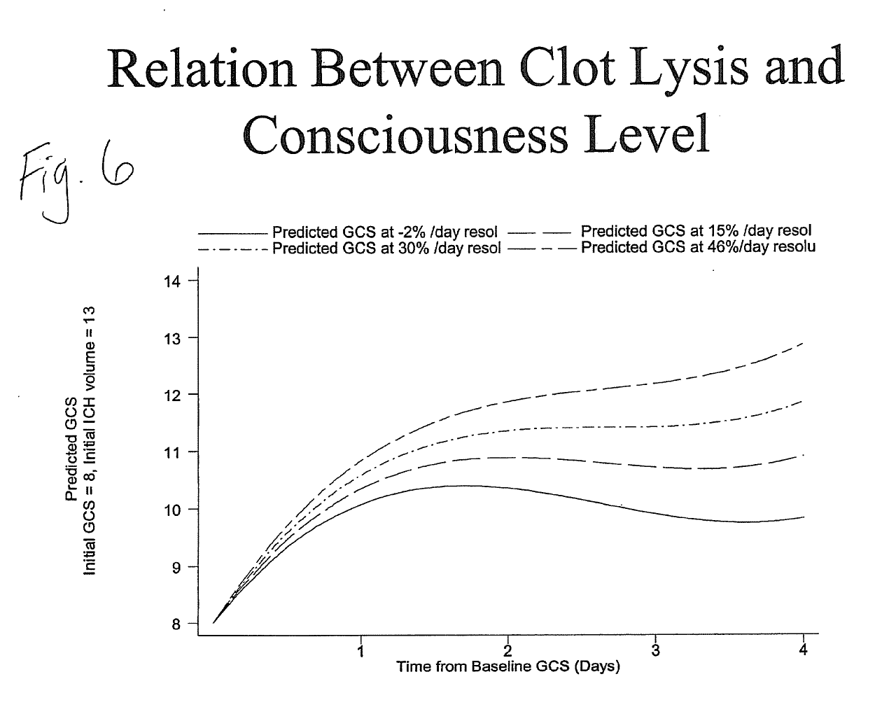

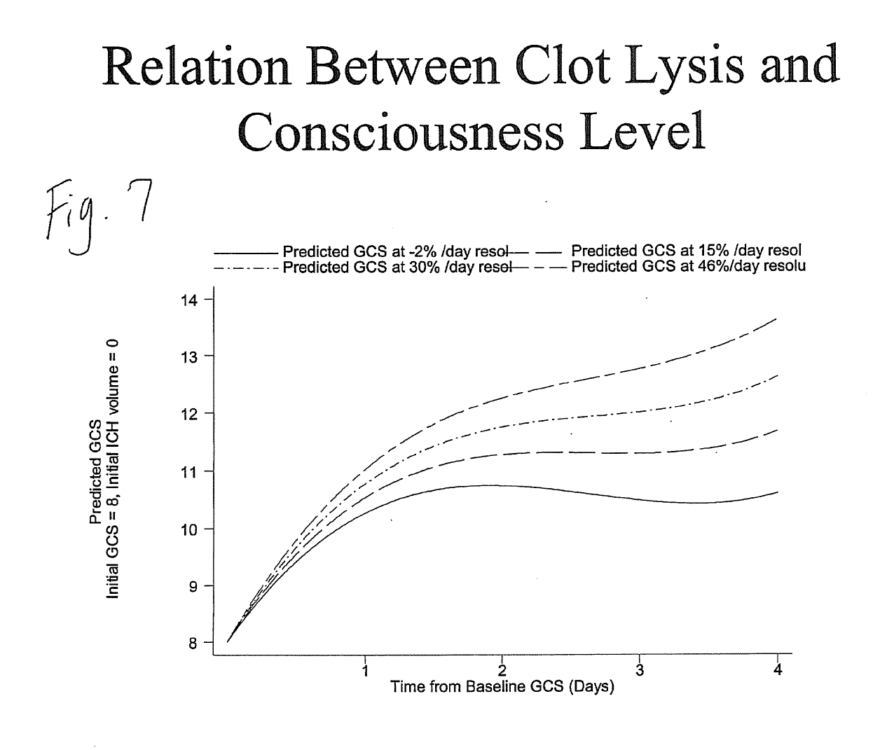

The effect of clot resolution was a one point improvement in GCS 96 hours after baseline for each 15% per day increase in clot resolution. Improvement in the level of consciousness after 96 hours for a patient with a clot resolving at the fastest rate observed would be 3.3 GCS points greater than that for a patient with a clot resolving at the slowest rate observed. FIG. 6 shows a statistical model for the relationship between clot lysis and consciousness level for patients with an initial ICH volume of 13. FIG. 7 shows the model for patients with an initial ICH volume of 0. Both of these models show that the faster the rate of clot resolution, the higher the predicted GCS score.

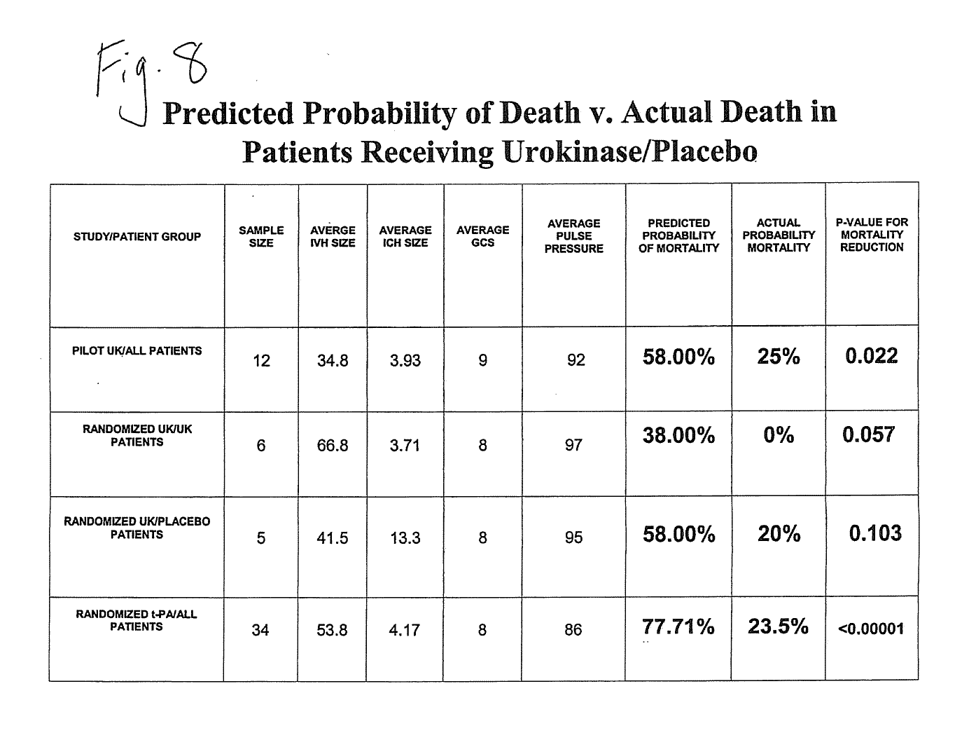

The mean calculated 30-day mortality and the mean actual 30-day mortality were compared using the exact hypothesis test for binomial random variables. The probability of obtaining the number of deaths observed, or fewer, under the predicted mortality rate, was calculated accepting statistical significance only if p<0.05. FIG. 8 shows a table comparing the data for several urokinase studies and the randomized, still blinded, rt-PA study.

### REFERENCES CITED

References citations throughout the specification (numerical superscripts) refer to the following references. All of the following references, as well as those cited directly in the specification, are incorporated herein in their entirety by this reference.

1. Adams H P, Torner J C, Kassell N F: Intraventricular hemorrhage among patients with recently ruptured aneurysms: a report of the Cooperative Aneurysm Study. Stroke 23:140, 1992.

2. Adams R E, Diringer M N: Response to external ventricular drainage in spontaneous intracerebral hemorrhage with hydrocephalus. Neurology 50:519-523, 1998.

3. Ahmann P A, Lazzarra A, Dykes F D: Intraventricular hemorrhage in the high risk preterm infant: incidence and outcome. Ann Neurol 7:118-124, 1980.

4. Akdemir H, Selcuklu A, Pasaoglu A: Treatment of severe intraventricular hemorrhage by intraventricular infusion of urokinase. Neurosurg Rev 18:95-100, 1995.

5. Alkjaersig N, Fletcher A P, Sherry S: The mechanism of clot dissolution by plasmin. J Clin Invest 38:1086-1095, 1959.

6. Asano T, Tanishima T, Sasaki T: Possible participation of free radical reactions initiated by clot lysis in the pathogenesis of vasospasm after subarachnoid hemorrhage. Baltimore: Williams & Wilkins, 1980.

7. Aucoin P J, Kotilainen B R, Gantz N M: Intracranial pressure monitors. Epidemiologic study of risk factors and infections. Am J Med 80:369-376, 1986.

8. Bagley C: Blood in cerebrospinal fluid. Resultant functional and organic alterations in the central nervous system. Arch Surg 17:39-81, 1928.

9. Bergstrom M, Ericson K, Levander B: Variation with time of the attenuation values of intracranial hematomas. J Comput Assist Tomography 1:57-63, 1977.

10. Broderick J P, Brott T, Tomsick T: Intracerebral hemorrhage more than twice as common as subarachnoid hemorrhage. J Neurosurg 78:188-191, 1993.

11. Broderick J P, Brott T G, Duldner J E, et al: Volume of intracerebral hemorrhage: a powerful and easy-to-use predictor of 30-day mortality. Stroke 24:987-993, 1993.

12. Broderick J P, Brott T G, Tomsick T, et al: Ultra-early evaluation of intracerebral hemorrhage. J Neurosurg 72:195-199, 1990.

13. Brott T, Thalinger K, Hertzberg V: Hypertension as a risk factor for spontaneous intracerebral hemorrhage. Stroke 17:1078-1083, 1986.

14. Cannon C P, Gibson C M, McCabe C H, et al: TNK-tissue plasminogen activator compared with front loaded alteplase in acute myocardial infarction results of the TIMI 10b trial. Circulation 98:2805-2814, 1998.

15. Conway J E, Oshiro E M, Piantadosi S: Ventricular blood is an admission CT variable which predicts poor clinical outcome after aneurysmal subarachnoid hemorrhage. American Association of Neurological Surgeons Annual Meeting, Philadelphia, Pa. J Neurosurg 88:398A, 1998.

16. Coplin W M, Vinas F C, Agris J M, et al: A cohort study of the safety and feasibility of intraventricular urokinase for nonaneurysmal spontaneous intraventricular hemorrhage. Stroke 29:1573-1579, 1998.

17. Daverat P, Castel J P, Dartigues J F: Death and functional outcome after spontaneous intracerebral hemorrahge. A prospective study of 166 cases using multivariate analysis. Stroke 22:1-6, 1991.

18. Deland F H, James A E, Jr., Ladd D J: Normal pressure hydrocephalus. A histologic study. Amer J Clin Path 58:58-63, 1972.

19. Diringer M N, Edwards D F, Zazulia A R: Hydrocephalus: a previously unrecognized predictor of poor outcome from supratentorial intracerebral hemorrhage. Stroke 29:1352-1357, 1998.

20. Ellington E, Margolis G: Block of arachnoid villus by subarachnoid hemorrhage. J Neurosurg 30:651-657, 1969.

21. Fennerty A G, Levine M N, Hirsh J: Hemorrhagic complications of thrombolytic therapy in the treatment of myocardial infarction and venous thromboembolism. Chest 95:88S-97S, 1989.

22. Findlay J M, Grace M G, Weir B K: Treatment of intraventricular hemorrhage with tissue plasminogen activator. Neurosurgery 32:941-947, 1993.

23. Findlay J M, Kassell N F, Weir B K: A randomized trial of intraoperative, intracisternal tissue plasminogen activator for the prevention of vasospasm. Neurosurgery 37:168-176, 1995.

24. Findlay J M, Weir B K, Gordon P: Safety and efficacy of intrathecal thrombolytic therapy in a primate model of cerebral vasospasm. Neurosurgery 24:491-498, 1989.

25. Findlay J M, Weir B K, Kanamaru K: Intrathecal fibrinolytic therapy after subarachnoid hemorrhage: dosage study in a primate model and review of the literature. Can J Neurol Sci 16:28-40, 1989.

26. Findlay J M, Weir B K, Kassell N F: Intracisternal recombinant tissue plasminogen activator after aneurysmal subarachnoid hemorrhage. J Neurosurg 75:181-188, 1991.

27. Findlay J M, Weir B K, Steinke D: Effect of intrathecal thrombolytic therapy on subarachnoid clot and chronic vasospasm in a primate model of SAH. J Neurosurg 69:723-735, 1988.

28. Findlay J M, Weir B K, Stollery D E: Lysis of intraventricular hematoma with tissue plasminogen activator. Case report. J Neurosurg 74:803-807, 1991.

29. Fisher C M, Kistler J P, Davis J M: Relation of cerebral vasospasm to subarchnoid hemorrhage visualized by computerized tomographic scanning. Neurosurgery 6:1-9, 1980.

30. Handa Y, Osaka K, Okamoto S: Breakdown products of erythrocytes as a cause of cerebral vasospasm. Baltimore: Williams & Wilkins, 1980.

31. Handa Y, Weir B K, Nosko M: The effect of timing of clot removal on chronic vasospasm in a primate model. J Neurosurg 67:558-564, 1987.

32. Hoffman R, Benz E J, Jr., Shattil S J: Hematology: Basic Principles and Practice, ed 2nd. New York: Churchill Livingstone, 1995.

33. Kane P J, Modha P, Strachan R D: The effect of immunosuppression on the development of cerebral oedema in an experimental model of intracerebral hemorrhage: whole body and regional inrradiation. J Neurol Neurosurg Psychiatry 55:781-786, 1992.

34. Kanter R K, Weiner L B, Patti M: Infectiuos complications and duration of intracranial pressure monitoring. Crit Care Med 13:837-839, 1985.

35. Kase C S, O'Neal A M, Fisher M: Intracranial hemorrhage after use of tissue plasminogen activator for coronary thrombolytics. Ann Intern Med 112:17-21, 1990.

36. Kase C S, Pessin M S, Zivin J A: Intracranial hemorrhage after coronary thrombolysis with tissue plasminogen activator. Am J Med 92:384-390, 1992.

37. Kase C S, Robinson R K, Stein R W: Anticoagulant-related intracerebral hemorrhage. Neurology 35:943-948, 1985.

38. Kassell N F, Torner J C, Adams H P, Jr.: Antifibrinolytic therapy in the acute period following aneurysmal subarachnoid hemorrhage. Preliminary observations from The Cooperative Aneurysm Study. J Neurosurg 61:225-230, 1984.

39. Kibler R F, Couch R S C, Crompton M R: Hydrocephalus in the adult following spontaneous hemorrhage. Brain 84:45-61, 1961.

40. Kodama N, Sasaki T, Kawakami M: Cisternal irrigation therapy with urokinase and ascorbic acid, in Sano K, Takakura K, Kassell N F (eds): Cerebral Vasospasm. Tokyo: University of Tokyo Press, 1990, pp 292-296.

41. Lee K S, Bae H G, Yun I G: Recurrent intracerebral hemorrhage due to hypertension. Neurosurgery 26:586-590, 1990.

42. LeMay M, Hochberg F H: Ventricular differences between hydrostatic hydrocephalus and hydrocephalus ex vacuo by computed tomography. Neurorad 17:191-195, 1979.

43. Lisk D R, Pasteur W, Rhoades H, et al: Early presentation of hemispheric intracerebral hemorrhage: prediction of outcome and guidelines for treatment allocation. Neurology 44:133-139, 1994.

44. Marder V J, Sherry S: Thrombolytic therapy. Current status: part 1. NEJM 318:1512-1520, 1988.

45. Marder V J, Sherry S: Thrombolytic therapy. Current status: part 2. NEJM 318:1585-1595, 1988.

46. Matsumoto K, Hondo H: CT-guided stereotaxic evacuation of hypertensive intracerebral hematomas. J Neurosurg 61:440-448, 1984.

47. Mayfrank L, Kissler J, Raoofi R: Ventricular dilatation in experimental intraventricular hemorrhage in pigs. Characterization of cerebrospinal fluid dynamics and the effects of fibrinolytic treatment. Stroke 28:141-148, 1997.

48. Mayfrank L, Lippitz B, Groth M: Effect of recombinant tissue plasminogen activator on clot lysis and ventricular dilatation in the treatment of severe intraventricular hemorrhage. Acta Neurochir 122:32-38, 1993.

49. Mayhall C G, Archer N H, Lamb V A: Ventriculostomy-related infections: a prospective epidemiologic study. NEJM 310:553-559, 1984.

50. Messina A V, Chernik N L: Computer tomography: the “resolving” intracerebral hemorrhage. Radiology 118:609-613, 1976.

51. Mizoi K, Yoshimoto T, Fujiwara S: Prevention of vasospasm by clot removal and intrathecal bolus injection of tissue-type plasminogen activator: preliminary report. Neurosurgery 28:807-813, 1991.

52. Mizoi K, Yoshimoto T, Takahashi A: Prospective study on the prevention of cerebral vasospasm by intrathecal fibrinolytic therapy with tissue-type plasminogen activator. J Neurosurg 78:430-437, 1993.

53. Mohr G, Ferguson G, Khan M: Intraventricular hemorrhage from ruptured aneurysm. J Neurosurg 58:482-487, 1983.

54. Montes J M, Wong J H, Fayad P B, et al: Stereotactic computed tomographic-guided aspiration and thrombolysis of intracerebral hematoma: protocol and preliminary experience. Stroke 31:834-840, 2000.

55. Morgenstern L B, Demchuk A M, Kim D G, et al: Rebleeding leads to poor outcome in ultra-early craniotomy for intracerebral hemorrhage. Neurology 56:1294-1299, 2001.

56. Naff N J, Carhuapoma J R, Williams M A, et al: Treatment of intraventricular hemorrhage with urokinase: effect on 30-day survival. Stroke 31:841-847, 2000.

57. Naff N J, Keyl P M, Tuhrim S, et al: Intraventricular thrombolysis speeds blood clot resolution: results of a randomized, double-blinded controlled clinical trial. Neurosurgery:submitted.

58. Naff N J, Williams M A, Rigamonti D R, et al: Blood clot resolution in human cerebrospinal fluid: evidence of first-order kinetics. Neurosurgery 49:614-619; discussion 619-621, 2001.

59. New P F, Aronow S: Attenuation measurements of whole blood and blood fractions in computed tomography. Radiology 121:635-640, 1976.

60. Pang D, Sclabassi R J, Horton J A: Lysis of intraventricular blood clot with urokinase in a canine model: part 1: canine intraventricular blood cast model. Neurosurgery 19:540-546, 1986.

61. Pang D, Sclabassi R J, Horton J A: Lysis of intraventricular blood clot with urokinase in a canine model: part 2: in vivo safety study of intraventricular urokinase. Neurosurgery 19:547-552, 1986.

62. Pang D, Sclabassi R J, Horton J A: Lysis of intraventricular blood clot with urokinase in a canine model: part 3: effects of intraventricular urokinase on clot lysis and posthemorrhagic hydrocephalus. Neurosurgery 19:553-572, 1986.

63. Papile L A, Burstein J, Burstein R: Incidence and evolution of subependymal and intraventricular hemorrhage: a study of infants with birth weights less than 1,500 gm. J Ped 92:529-534, 1978.

64. Peterson E W, Choo S H, Lewis A J: Lysis of blood clot and experimental treatment of subarachnoid hemorrhage. Baltimore: Williams & Wilkins, 1980.

65. Rakoczi I, Wiman B, Collen D: On the biological significance of the specific interaction between fibrin, plasminogen and antiplasmin. Biochim Biophys Acta 540:295-300, 1978.

66. Rohde V, Schaller C, Hassler W E: Intraventricular recombinant tissue plasminogen activator for lysis of intraventricular haemorrhage. J Neurol Neurosurg Psychiatry 58:447-451, 1995.

67. Rosner M J, Becker D P: ICP monitoring: complications and associated factors. Clin Neurosurg 23:494-519, 1976.

68. Sasaki T, Ohta T, Kikuch H: Preliminary clinical trial of intrathecal rt-PA (TD-2061) for the prevention of cerebral vasospasm in patients with aneurysmal subarachnoid hemorrahge. No To Shinkei—Brain & Nerve 44:1001-1008, 1992.

69. Schultz M, Moore K, Foote A W: Bacterial ventriculitis and duration of ventriculostomy catheter insertion. J Neurosci Nurs 25:158-164, 1993.

70. Schwartz S, Schwab S, Steiner H H, et al: Fibrinolysis of intraventricular hematoma with rt-PA. Nervenarzt 70:123-130, 1999.

71. Scott W R, New P F, Davis K R: Computerized axial tomography of intracerebral and intraventricular hemorrhage. Radiology 112:73-80, 1974.

72. Shapiro S A, Campbell R L, Scully T: Hemorrhagic dilation of the fourth ventricle: an ominous predictor. J Neurosurg 80:805-809, 1994.

73. Shen P H, Matsuoka Y, Kawajiri K: Treatment of intraventricular hemorrhage using urokinase. Neurol Med Chir (Tokyo) 30:329-333, 1990.

74. Steiner L, Bergvall U, Zwetnow N: Quantitative estimation of intracerebral and intraventricular hematoma by computer tomography. Acta Radiol Suppl 346:143-154, 1975.

75. Steinke W, Sacco R L, Mohr J P: Thalamic stroke. Presentation and prognosis of infarcts and hemorrhages. Arch Neurol 49:703-710, 1992.

76. Todo T, Usui M, Takakura K: Treatment of severe intraventricular hemorrhage by intraventricular infusion of urokinase. J Neurosurg 74:81-86, 1991.

77. Tuhrim S, Dambrosia J M, Price T R: Intracerebral hemorrhage: external validation and extension of a model for prediction of 30-day survival. Ann Neurol 29:658-663, 1991.

78. Tuhrim S, Horowitz D R, Sacher M, et al: Validation and comparison of models predicting survival following intracerebral hemorrhage. Crit Care Med 23:950-954, 1995.

79. Tuhrim S, Horowitz D R, Sacher M, et al: Volume of ventricular blood is an important determinant of outcome in supratentorial intracerebral hemorrhage. Crit Care Med 27:617-621, 1999.

80. Usui M, Saito N, Hoya K: Vasospasm prevention with postoperative intrathecal thrombolytic therapy: a retrospective comparison of urokinase, tissue plasminogen activator, and cisternal drainage alone. Neurosurgery 34:235-245, 1994.

81. Yasargil M G, Yonekawa Y, Zumstein B: Hydrocephalus following spontaneous subarachnoid hemorrhage. Clinical features and treatment. J Neurosurg 39:474-479, 1973.

82. Young W B, Lee K P, Pessin M S: Prognostic significance of ventricular blood in supratentorial hemorrhage: a volumetric study. Neurology 40:616-619, 1990.

83. Zabramski J M, Spetzler R F, Lee K S: Phase I trial of tissue plasminogen activator for the prevention of vasospasm in patients with aneurysmal subarachnoid hemorrhage. J Neurosurg 75:189-196, 1991.

84. Akdemir H, Selcuklu A, Pasaoglu A, Oktem I S, Kavuncu I: Treatment of severe intraventricular hemorrhage by intraventricular infusion of urokinase. Neurosurg Rev 18:95-100, 1995.

85. Coplin W M, Vinas F C, Agris J M, Buciuc R, Michael D B, Diaz F G, Muizelaar J P: A cohort study of the safety and feasibility of intraventricular urokinase for nonaneurysmal spontaneous intraventricular hemorrhage. Stroke 29:1573-1579, 1998.

86. Daverat, P. et al. (1991) Stroke 22:1-6.

87. Dawson-Saunders, B. and Trapp, R. G.: Basic and Clinical Biostatistics. Norwalk, Appleton and Lange, 1994.

88. Fang, J. and Alderman M. H. (2001) Stroke 32:2221-2226.

89. Findlay J M, Grace M G, Weir B K: Treatment of intraventricular hemorrhage with tissue plasminogen activator. Neurosurgery 32:941-947, 1993.

90. Findlay J M, Weir B K, Stollery D E: Lysis of intraventricular hematoma with tissue plasminogen activator. Case report. J Neurosurg 74:803-807, 1991.

91. Goh K Y, Poon W S: Recombinant tissue plasminogen activator for the treatment of spontaneous adult intraventricular hemorrhage. Surg Neurol 50:526-531, 1998.

92. Grabb P A: Traumatic intraventricular hemorrhage treated with intraventricular recombinant-tissue plasminogen activator: technical case report. Neurosurgery 43:966-969, 1998.

93. Haines S J, Lapointe M: Fibrinolytic agents in the treatment of intraventricular hemorrhage in adults. Crit Rev Neurosurg 8:169-175, 1998.

94. Hansen A R, Volpe J J, Goumnerova L C, Madsen J R: Intraventricular urokinase for the treatment of posthemorrhagic hydrocephalus. Pediatr Neurol 17:213-217, 1997.

95. Hudgins R J, Boydston W R, Hudgins P A, Adler S R: Treatment of intraventricular hemorrhage in the premature infant with urokinase. A preliminary report. Pediatr Neurosurg 20:190-197, 1994.

96. Hudgins R J, Boydston W R, Hudgins P A, Morris R, Adler S M, Gilreath C L: Intrathecal urokinase as a treatment for intraventricular hemorrhage in the preterm infant. Pediatr Neurosurg 26:281-287, 1997.

97. Mayfrank L, Hutter B O, Kohorst Y, Kreitschmann-Andermahr I, Rohde V, Thron A, Gilsbach T M: Influence of intraventricular hemorrhage on outcome after rupture of intracranial aneurysm. Neurosurg Rev 24:185-191, 2001.

98. Mayfrank L, Kissler J, Raoofi R, Delsing P, Weis J, Kuker W, Gilsbach J M: Ventricular dilatation in experimental intraventricular hemorrhage in pigs. Characterization of cerebrospinal fluid dynamics and the effects of fibrinolytic treatment. Stroke 28:141-148, 1997.

99. Mayfrank L, Lippitz B, Groth M, Bertalanffy H, Gilsbach J M: Effect of recombinant tissue plasminogen activator on clot lysis and ventricular dilatation in the treatment of severe intraventricular haemorrhage. Acta Neurochir 122:32-38, 1993.

100. Mayfrank L, Rohde V, Gilsbach J M: Fibrinolytic treatment of intraventricular haemorrhage preceding surgical repair of ruptured aneurysms and arteriovenous malformations. Br J Neurosurg 13:128-131, 1999.

101. Murry K R, Rhoney D H, Coplin W M: Urokinase in the treatment of intraventricular hemorrhage. Ann Pharmacother 32:256-258, 1998.

102. Naff N J, Bryan R N, Rigamonti D R, Hanley D F: Blood clot resolution in human CSF: A serial quantitative study. Presented at Joint Section on Cerebrovascular Surgery Annual Meeting, Anaheim, Calif., 1997.

103. Naff N J, Carhuapoma J R, Williams M A, Bhardwaj A, Ulatowski J A, Bederson J, Bullock R, Schmutzhard E, Pfausler B, Keyl P M, Tuhrim S, Hanley D F: Treatment of intraventricular hemorrhage with urokinase: effects on 30-Day survival. Stroke 31:841-847, 2000.

104. Naff N J, Williams M A, Rigamonti D, Keyl P M, Hanley D F: Blood clot resolution in human cerebrospinal fluid: evidence of first-order kinetics. Neurosurgery 49:614-619, 2001.

105. Nieuwkamp D J, de Gans K, Rinkel G J, Algra A: Treatment and outcome of severe intraventricular extension in patients with subarachnoid or intracerebral hemorrhage: a systematic review of the literature. J Neurol 247:117-121, 2000.

106. Pang D, Sclabassi R J, Horton J A: Lysis of intraventricular blood clot with urokinase in a canine model: Part 3. Effects of intraventricular urokinase on clot lysis and posthemorrhagic hydrocephalus. Neurosurgery 19:553-572, 1986.

107. Rainov N G, Burkert W L: Urokinase infusion for severe intraventricular haemorrhage. Acta Neurochir 134:55-59, 1995.

108. Rhoney D H, Coplin W M, Zaran F K, Brish L K, Weingarten C M: Urokinase activity after freezing: implications for thrombolysis in intraventricular hemorrhage. Am J Health Syst Pharm 56:2047-2051, 1999.

109. Schwarz S, Schwab S, Steiner H H, Hacke W: Secondary hemorrhage after intraventricular fibrinolysis: a cautionary note: a report of two cases. Neurosurgery 42:659-662, 1998.

110. Schwarz S, Schwab S, Steiner H H, Hanley D, Hacke W: [Fibrinolysis of intraventricular hematoma with rt-PA]. Nervenarzt 70:123-130, 1999.

111. Shen P H, Matsuoka Y, Kawajiri K, Kanai M, Hoda K, Yamamoto S, Nishimura S: Treatment of intraventricular hemorrhage using urokinase. Neurol Med Chir (Tokyo) 30:329-333, 1990.

112. Steiner L, Bergvall U, Zwetnow N: Quantitative estimation of intracerebral and intraventricular hematoma by computer tomography. Acta Radiol Suppl 346:143-154,1975.

113. Todo T, Usui M, Takakura K: Treatment of severe intraventricular hemorrhage by intraventricular infusion of urokinase. J Neurosurg 74:81-86, 1991.

114. Tuhrim S, Horowitz D R, Sacher M, Godbold J H: Volume of ventricular blood is an important determinant of outcome in supratentorial intracerebral hemorrhage. Crit Care Med 27:617-621, 1999.

115. Tung M Y, Ong P L, Seow W T, Tan K K: A study on the efficacy of intraventricular urokinase in the treatment of intraventricular haemorrhage. Br J Neurosurg 12:234-239, 1998.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method for the prevention or treatment of an extravascular hematoma or blood clot in a subject, comprising administering to the subject a therapeutically effective amount of a thromoblytic agent, thereby preventing or treating the extravascular hematoma or blood clot.

2. The method of claim 1, wherein the blood clot is associated with intraventriclar hemorrhage.

3. The method of claim 2, wherein the blood clot is further associated with intracerebral hemorrhage.

4. The method of claim 1, where in the blood clot is associated with subarachnoid hemorrhage.

5. The method of any of claims 1-4, wherein the thrombolyic agent is urokinase.

6. The method of any of claims 1-4, wherein the thrombolytic agent is t-PA or rt-PA.

7. The method of any of claims 1-6, wherein the thrombolytic agent is administered in conjunction with EVD.

8. The method of any of claims 1-7, wherein the thrombolytic agent is first administered between about 12-24 hours after diagnosis of intraventricular hemorrhage, intracerebral hemmorhage, and/or subarachnoid hemorrhage.

9. The method of any of claims 1-7, wherein the thrombolytic agent is first administered between about 24-48 hours after diagnosis of intraventricular hemorrhage, intracerebral hemmorhage, and/or subarachnoid hemorrhage.

10. The method of any of claims 1-9, further comprising performing CT scans at intervals of about 6-24 hours to monitor blood clot size and/or monitor whether bleeding is occurring.

11. The method of any of claims 1-10, wherein the thrombolytic agent is administered at least about every 4 hours.

12. The method of any of claims 1-11, wherein the thrombolytic agent is administered at least about every 6 hours.

13. The method of any of claims 1-12, wherein the thrombolytic agent is administered at least about every 8 hours.

14. The method of any of claims 1-13, wherein the thrombolytic agent is administered at least about every 10 hours.

15. The method of any of claims 1-14, wherein the thrombolytic agent is administered at least about every 12 hours.

16. The method of any of claims 1-15, wherein administration of the thrombolytic agent is stopped when the blood clot size is about 80% of its original size.

17. The method of claim 16, wherein the blood clot reaches 80% of its original size about 3 days after the first administration of the thrombolytic agent.

18. The method of any of claim 5 or 7-17, wherein the urokinase is administered in doses of about 5000-50,000 units.

19. The method of claim 18, wherein the urokinase is administered in doses of about 12,500 units.

20. The method of any of claims 6-17, wherein the t-PA or rt-PA is administered in doses of about 0.1-10 mg.

21. The method of claim 20, wherein the t-PA or rt-PA is administered in doses of about 3 mg.
